(19) United States
(12) Patent Application Publication
(10) Pub. No.: US 2024/0209446 A1
(43) Pub. Date: Jun. 27, 2024

(54) CIRCULATING NONCODING RNAS AS A SIGNATURE OF AUTISM SPECTRUM DISORDER SYMPTOMATOLOGY

(71) Applicant: Qatar Foundation for Education, Science and Community Development, Doha (QA)

(72) Inventors: Sara ABDULLA, Doha (QA); Salam SALLOUM-ASFAR, Doha (QA)

(21) Appl. No.: 18/288,778

(22) PCT Filed: Apr. 28, 2022
(86) PCT No.: PCT/QA2022/050007

§ 371 Date: Oct. 27, 2023

Related U.S. Application Data
(60) Provisional application No. 63/180,952, filed on Apr. 28, 2021.

(51) Int. Cl. C12Q 1/6883; C12Q 1/6874

(52) U.S. Cl. CPC C12Q 1/6883; C12Q 1/6874; C12Q 2600/178

(57) ABSTRACT

The present specification provides assays for diagnosing and stratifying Autism Spectrum Disorder based on profiles of circulating noncoding RNAs.

### BACKGROUND

Autism Spectrum Disorder (ASD) is a multi-faceted neurodevelopmental disorder that manifests during the early years of child development. The complexity of ASD makes clinically diagnosing the condition difficult. Although awareness of the complex heterogeneity of ASD has increased, and continues to, there is still little known about the etiology and pathophysiology of the disorder. Current classifications of individuals with ASD house them under two main umbrella categories; communication, and social interactions/behaviors.

To date, subjective and clinical diagnosis has been the common method of identifying children with the disorder, which although helpful, is still far from ideal. This method risks late/missed diagnoses and ineffective therapeutic interventions. Thus, methods to objectively and systematically identify children with ASD are lacking.

### SUMMARY

Differences in the amounts of various noncoding RNA molecules in the blood circulation (cir-ncRNA) of children with ASD have been found between those who are severely and mildly affected with the disorder. Expression level profiles for sets of the RNAs can thus be used in the diagnosis and stratification of ASD, in either a prospective or confirmatory manner.

The expression level profiles of cir-ncRNA may be based on the expression levels of:
a. microRNA (miRNA);
b. piwi-interacting RNA (piRNA);
c. small nucleolar RNA (snoRNA); or
d. Y-RNA molecules; and
e. combinations thereof.

Differential expression, as measured in the circulation, of 100 miRNAs, 29 piRNAs, 23 snoRNAs, and 4 Y-RNAs between subjects with severe and mild symptoms of ASD, is disclosed.

In various embodiments, measuring expression levels in circulation entails analysis of a sample of whole blood, plasma, serum, or combinations thereof.

### BRIEF DESCRIPTION OF THE DRAWINGS

FIG. 1 presents an overview of the experimental design of the study. (1) Sample selection; (2) Phlebotomy; (3) Blood fractionation; (4) RNA extraction and elimination of contaminants; (5) Assessment of isolated RNA and quality check using qRT-PCR; (6) small library preparation; (7) Library quantification and assessment using bioanalyzer and Qubit; (8) Sequencing of libraries using NGS technology HiSeq 3000/4000 Illumina sequencing system; (9) BCL2 to Fastaq conversion and generation of Fastqc files. cDNA synthesis for small RNA without fragmentation; (10) Fastaq reads; (11) Data analysis using CLC Genomics Workbench program version 20.0.4 and Geneglobe Data analysis center.

FIG. 2 depicts circulating transcriptome profile analysis on plasma. The pie charts represent the relative abundance of families of RNA present in the plasma of children that manifest severe symptoms of ASD (N=22) and mild symptoms of ASD (N=23). The group labeled as “Other RNA” in the pie charts is representative of reads derived from several Gencode annotation categories such as snoRNAs, YRNAs, etc.

FIG. 3 depicts miRNA expression analysis using CLC Genomics Workbench v20.0.4. (FIG. 3A) QIAseq miRNA Differential Expression workflow. The workflow calculates differential expressions for expression tables using multi-factorial statistics. Results are grouped in mature and in seed expression tables that can be used for differential expression analysis. (FIG. 3B) Global views of gene expression utilizing the Principal Component Analysis (PCA) software between subjects that manifest severe symptoms of ASD (purple dots), and mild symptoms of ASD (yellow dots) samples. The analysis was performed by the CLC Genomic Workbench software using default setting that includes a threshold to remove low background level intensities. PCA percent mapping on the top of the plot indicates the explained variability on the first coordinates. (FIG. 3C) Hierarchical clustering analysis of miRNA expression profile. Two-dimensional heat map of expression values. Each column corresponds to one sample, and each row corresponds to a miRNA. The samples and features are both hierarchically clustered. (FIG. 3D) Top differentially expressed miRNAs in severe ASD cases (Log₂ fold change >2; p<0.05).

FIG. 4 depicts most highly rated network through IPA analysis. The network representation of the most highly rated network (Cancer, Organismal Injury and Abnormalities, Reproductive System Disease). The genes that are shaded were determined to be significant from the statistical analysis. The genes shaded red are upregulated and those that are green are downregulated. The intensity of the shading shows to what degree each gene was up or downregulated. A solid line represents a direct interaction between the two gene products and a dotted line means there is an indirect interaction.

FIG. 5 depicts piRNA expression analysis using CLC Genomics Workbench v20.0.4. (FIG. 5A) Global views of gene expression, utilizing the Principal Component Analysis (PCA) software, between subjects that manifested severe symptoms of ASD (purple dots), and mild symptoms of ASD (yellow dots) samples. The analysis was performed by the CLC Genomic Workbench software using default setting that includes a threshold to remove low background level intensities. PCA percent mapping on the top of the plot indicates the explained variability on the first coordinates. (FIG. 5B) Hierarchical clustering analysis of piRNA expression profile. Two-dimensional heat map of expression values. Each column corresponds to one sample, and each row corresponds to a piRNA. (FIG. 5C) Top differentially expressed piRNAs in severe ASD cases (Log₂ fold change >2; p<0.05).

FIG. 6 depicts snoRNAs and Y-RNAs DE expression analysis. (FIG. 6A) Global views of gene expression, utilizing the Principal Component Analysis (PCA) software, between subjects that manifested ASD severe symptoms (purple dots), and ASD mild symptoms (yellow dots) samples. The analysis was performed by the CLC Genomic Workbench software using default setting that 801 includes a threshold to remove low background level intensities. PCA percent mapping on the top of the plot indicates the explained variability on the first coordinates. (FIG. 6B) Hierarchical clustering analysis of snoRNAs and Y-RNAs expression profile. Two-dimensional heat map of expression values. Each column corresponds to one sample, and each row corresponds to a snoRNA or Y-RNA. (FIG. 6C) Top differentially expressed snoRNAs and Y-RNAs in severe ASD cases (Log₂ fold 806 change >2; p<0.05).

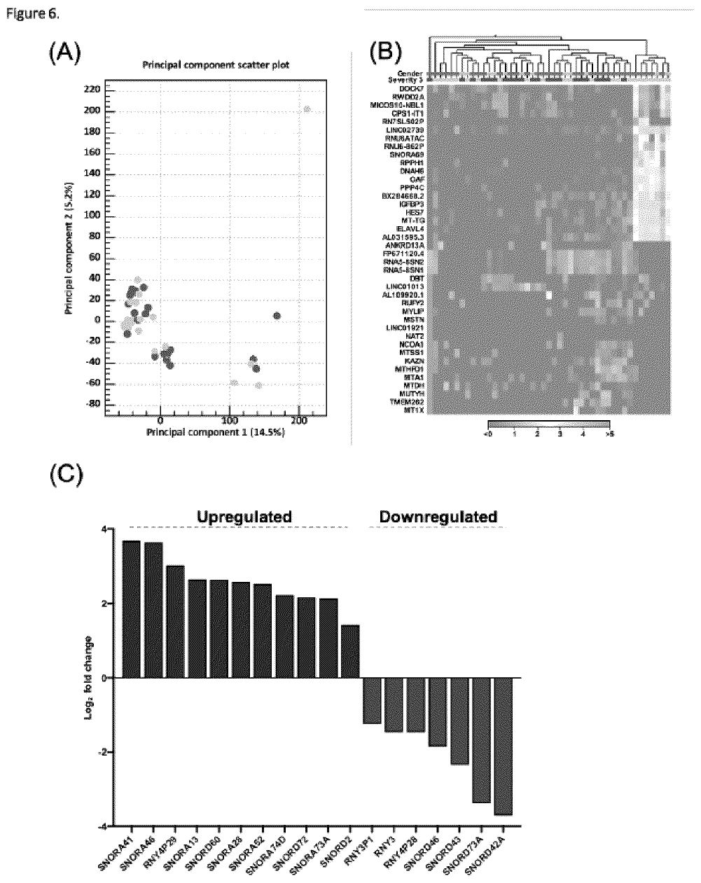

### DESCRIPTION

ASD is a developmental disease and it is conceivable, even probable, that cir-ncRNA profiles could change with development of the disorder. Clinically, there is the greatest need/benefit to diagnose and stratify ASD in younger children. The herein disclosed ncRNA profiles were obtained from plasma samples from children with a median age of about 7.6 years (see Table 4, below). Thus, in various embodiments, the methods of determining a cir-nrRNA profile are carried out on children ≤10 years of age, ≤8 years of age, from 5-10 years of age, or from 6-9 years of age.

If a child's assessed cir-ncRNA levels match a severe ASD cir-ncRNA profile, the child can be provided treatment appropriate for severe ASD. If a child's assessed cir-ncRNA levels match a mild ASD cir-ncRNA profile, the child can be provided treatment appropriate for mild ASD. If neither profile is matched at >90% of the ncRNA in the panel, in some embodiments, a fresh sample is obtained and evaluated using a more sensitive methodology, for example, qPCR.

Accurate and early diagnosis and stratification of Autism Spectrum Disorder (ASD) patients would facilitate timely intervention so that the adverse developmental trajectories and characteristic debilities associated with it could be mitigated or avoided. A reliable biomarker for the precise diagnosis and stratification of ASD has been lacking.

Consequently, ASD is identified mainly through behavioral phenotypes and characteristics. This subjective analysis leaves room for misdiagnosis, and potentially ineffective treatment strategies. Here we disclose biomarkers, specifically circulating noncoding RNAs (ncRNA) and panels thereof, which can be reliably used to provide objective identification of ASD and to better help stratify ASD cases within the spectrum to deliver more effective therapies.

Circulating ncRNAs have recently been categorized as potential diagnostic markers for various conditions, including neurological disorders. Although there have been studies associating circulating miRNAs to ASD, they have had various drawbacks including looking at older patients and using normal subjects as controls, which confounds the signals from patients residing in different positions along the spectrum. These drawbacks can obscure signals present in only a particular part of the spectrum and impair stratification. Other noncoding RNAs have not been studied at all, nor has isolation of circulating ncRNA from plasma been carried out.

As disclosed herein, the populations of four biotypes of circulating ncRNA in plasma (miRNA (the most abundant), piRNA, snoRNA, and Y-RNA) were examined as potentially containing biomarkers associated with ASD, and particularly with severe or mild ASD. Each group of subjects (with severe symptoms vs. mild symptoms) appeared to have apparent differences in circulating ncRNAs expression profiles. In particular, within the miRNA family, miR-302, which displayed substantially high read counts, we observed that hsa-miR-302a-5p, hsa-miR-302c-3p, hsa-miR-302a-3p, hsa-miR-302d-3p, hsa-miR-302b-3p, hsa-miR-302c-5p and hsa-miR-302b-5p were expressed at significantly high levels in cases of individuals that exhibited severe symptoms of ASD compared to those that expressed few or mild forms of ASD's defining characteristics.

Disclosed embodiments comprise determining an expression profile of circulating miRNAs differentially expressed between severe and mild ASD patients. In some embodiments, the miRNAs are a subset of the miRNAs of Tables 7 and/or 8 (see Example 2, below). In some embodiments, the expression profile is determined by quantitating the level of a predetermined panel of miRNAs selected from Tables 7 and/or 8. In some embodiments, level of expression is determined by deep sequencing. In some embodiments, expression level determined by deep sequencing is reported as reads per million (RPM), that is, how many times a particular sequence is detected per million RNA molecules sequenced. In some embodiments, the profile is associated with severe ASD. In some embodiments, the subset of miRNA from Tables 7 and/or 8 comprises the panel of Table 1.

TABLE 1
A panel of circulating miRNA for identifying severe ASD

| miRNA | | |
|---|---|---|
| # | ID | Sequence |
| 1 | hsa-miR-302a- | ACUUAAACGUGGAUGUACUUGCU (SEQ ID NO: 86) |
| | 5p | |
| 2 | hsa-miR-302c- | UAAGUGCUUCCAUGUUUCAGUGG (SEQ ID NO: 87) |
| | 3p | |
| 3 | hsa-miR-302a- | UAAGUGCUUCCAUGUUUUGGUGA (SEQ ID NO: 88) |
| | 3p | |
| 4 | hsa-miR-302d- | UAAGUGCUUCCAUGUUUGAGUGU (SEQ ID NO: 89) |
| | 3p | |
| 5 | hsa-miR-302b- | UAAGUGCUUCCAUGUUUUAGUAG (SEQ ID NO: 90) |
| | 3p | |
| 6 | hsa-miR-302c- | UUUAACAUGGGGGUACCUGCUG (SEQ ID NO: 91) |
| | 5p | |
| 7 | hsa-miR-135b- | UAUGGCUUUUCAUUCCUAUGUGA (SEQ ID NO: 92) |
| | 5p | |
| 8 | hsa-miR-373- | GAAGUGCUUCGAUUUUGGGGUGU (SEQ ID NO: 93) |
| | 3p | |
| 9 | hsa-miR-372- | AAAGUGCUGCGACAUUUGAGCGU (SEQ ID NO: 94) |
| | 3p | |
| 10 | hsa-miR-187- | UCGUGUCUUGUGUUGCAGCCGG (SEQ ID NO: 95) |
| | 3p | |
| 11 | hsa-miR-4745- | UGAGUGGGGCUCCCGGGACGGCG (SEQ ID NO: 96) |
| | 5p | |
| 12 | hsa-miR-184 | UGGACGGAGAACUGAUAAGGGU (SEQ ID NO: 97) |
| 13 | hsa-miR-219a- | UGAUUGUCCAAACGCAAUUCU (SEQ ID NO: 98) |
| | 5p | |
| 14 | hsa-miR-6516- | UUUGCAGUAACAGGUGUGAGCA (SEQ ID NO: 99) |
| | 5p | |
| 15 | hsa-miR-5189- | UCUGGGCACAGGCGGAUGGACAGG (SEQ ID NO: 100) |
| | 5p | |
| 16 | hsa-miR-378g | ACUGGGCUUGGAGUCAGAAG (SEQ ID NO: 101) |
| 17 | hsa-let-7f-2-3p | CUAUACAGUCUACUGUCUUUCC (SEQ ID NO: 102) |
| 18 | hsa-miR-6509- | AUUAGGUAGUGGCAGUGGAAC (SEQ ID NO: 103) |
| | 5p | |

In embodiments, severe ASD is associated with >300 RPM for miRNAs #1-10 and <10 RPM for miRNAs #11-18. In some embodiments, it is determined if each of these miRNA are present at these levels in a plasma sample from a child; that is, does the child's sample match the severe ASD profile? Some embodiments further comprise treating the child for ASD, such as severe ASD if their sample matches the severe ASD profile for these cir-ncRNA.

Further embodiments comprise determining an expression profile of circulating piRNAs differentially expressed between severe and mild ASD. In some embodiments, the piRNAs are a subset of the piRNAs of Table 11 (see Example 5, below). In some embodiments, the profile is determined by quantitating the level of a predetermined panel of piRNAs selected from Table 5. In some embodiments, level of expression is determined by deep sequencing. In some embodiments, expression level determined by deep sequencing is reported as reads per million (RPM), that is, how many times a particular sequence is detected per million RNA molecules sequenced. In some embodiments, the profile is associated with severe ASD. In some embodiments, the subset of piRNA from Table 11 comprises the panel of Table 2.

TABLE 2
A panel of circulating piRNA for identifying severe ASD

| piRNA | | |
|---|---|---|
| # | Name | Sequences |
| 1 | piR-hsa-22380 | TGTTAACCGAAAGATTGGTGGTTCGAG (SEQ ID NO: 104) |
| 2 | piR-hsa-28131 | GGCATTGGTGGTTCAGTGGTAGAATTCTCGC (SEQ ID NO: 105) |
| 3 | piR-hsa-27134 | GCCTGGATAGCTCAGTTGGTAGAGCATCAGA (SEQ ID NO: 106) |
| 4 | piR-hsa-28877 | GTTTCCGTAGTGTAGTGGTCATCACGTTCGCC (SEQ ID NO: 107) |
| 5 | piR-hsa-32221 | TAGTGCGCTATGCCGATCGGGTGTC (SEQ ID NO: 108) |
| 6 | piR-hsa-32184 | AGTGCGCTATGCCGATCGGGTGTCC (SEQ ID NO: 109) |
| 7 | piR-hsa-27493 | GCATTGGTGGTTCAGTGGTAGAATTCTCAC (SEQ ID NO: 110) |

In embodiments, severe ASD is associated with >200 RPM for each of piRNAs #1-7. In some embodiments, it is determined if each of these piRNA are present at these levels in a plasma sample from a child; that is, does the child's sample match the severe ASD profile? Some embodiments further comprise treating the child for ASD, such as severe ASD if their sample matches the severe ASD profile for these cir-ncRNA.

Further embodiments comprise determining an expression profile of circulating Y-RNAs and snoRNAs differentially expressed between severe and mild ASD. In some embodiments, the miRNAs comprise a subset of the Y-RNAs and snoRNAs of Table 12 (see Example 6, below). In some embodiments, the profile is determined by quantitating the level of a predetermined panel of Y-RNAs and/or snoRNAs selected from Table 12.

In some embodiments, the level of expression is determined by deep sequencing. In some embodiments, expression level determined by deep sequencing is reported as reads per million (RPM), that is, how many times a particular sequence is detected per million RNA molecules sequenced. In some embodiments, the profile is associated with severe ASD. In some embodiments, the subset of Y-RNAs and snoRNAs from Table 12 comprises the panel of Table 3.

TABLE 3
A panel of circulating Y-RNA and snoRNA for identifying severe ASD

| ncRNAs | | |
|---|---|---|
| # | Name | Sequence |
| 1 | RNY4P29 | GGUUGGUCCAAUGAUAGUGGGUUAUCAGAACUUAUUAACAU |
| | | UUAGUGUCACUAAAGUUGGUAUAAAACCCUCCACUGCUAAA |
| | | UUUAAAUGGUUUAUA (SEQ ID NO: 111) |
| 2 | SNORD2 | AAGUGAAAUGAUGGCAAUCAUCUUUCGGGACUGACCUGAAA |
| | | UGAAGAGAAUACUCAUUGCUGAUCACUU (SEQ ID NO: 112) |
| 3 | SNORD101 | GUUUGAAUGAUGACUUUAAUUGUCGGAUACCCCUUCACUCC |
| | | UUUUAUGAGUGAAACAUAAGAGUCUGACAAAC (SEQ ID NO: |
| | | 113) |
| 4 | SNORA46 | AGCACUAUAUUUAAACCUGUGGAUGGGAAUAUUCCCCAUUC |
| | | UUGGUUACGCUGUAGUGCAAAAGAAUUCCUGGCUCUCUGUU |
| | | GCACAGCUGACUUGUGCCAUUCUGCUGUUGCUGUAUAGAGU |
| | | UAAGGAACAUGG (SEQ ID NO: 114) |
| 5 | SNORA69 | AAAGCAGGUUGCAAUUACAGUGCUUCAUUUUGUGGAAGUAC |
| | | UGCCAUUAUCCUGCUGAAAGAAAAGCCGUGUUAAUCAUUUU |
| | | UGAUUUUGCCUUUAUGAGGGUAAAAUCAUGACAGAUUGACA |
| | | UGGACAAUU (SEQ ID NO: 115) |

In embodiments, severe ASD is associated with >100 RPM for ncRNA #1 and >200 RPM for ncRNAs 2-5. In some embodiments, it is determined if each of these Y-RNA or snoRNA are present at these levels in a plasma sample from a child; that is, does the child's sample match the severe ASD profile. Some embodiments further comprise treating the child for severe ASD if their sample matches the severe ASD profile for these cir-ncRNA.

Some embodiments of the above aspects further comprise a profile match confirmation step. In some embodiments, the profile match confirmation step comprises quantitative RT-PCT (qRT-PCR) of the ncRNA in the panel, for example, the panel of Tables 1, 2, or 3. In some embodiments, the profile match is considered confirmed if the fold-change by qRT-PCT is >2 for each ncRNA in the panel, as compared to a normal control.

It has been shown previously that the miR-302 family is critical in stem cell pluripotency and renewal and somatic cell DNA demethylation. We further performed pathway enrichment analysis to better understand miRNA's biological implications in the context of the regulatory system. Building on our observation of the large number of pathways enriched with ASD genes, we gained new insight into the interpretation of the underlying molecular mechanisms in ASD. Several factors contribute to the onset of ASD. Genetic association studies have shown how mutations in some genes can determine the onset of ASD phenotypes, including Phosphatase and tensin homolog protein (PTEN) and B-Raf Proto-Oncogene, Serine/Threonine kinase (BRAF). PTEN and BRAF are essential in synaptic transmission and plasticity and neuronal function and development of learning/memory. Thus there is an apparent association between the identified miRNA biomarkers and the pathophysiology of ASD.

miR-135b-5p is another miRNA that has been expressed at high levels in severe cases versus the mild ones. It has been previously described that variable regulation of DISC1 (Disrupted in schizophrenia 1) by miR-135b-5p in the brain may predispose to neuropsychiatric phenotypes. Furthermore, a recent study has shown that miR-135 can serve as a biomarker of Post-traumatic stress disorder (PTSD) and might be an important therapeutic target for dampening persistent and stress-enhanced memory. Thus, there is a plausible association of this biomarker with the pathophysiology of ASD as well.

It is widely known that besides miRNAs, other ncRNAs such as PIWI-interacting RNAS (piRNAs) act as key elements in cellular homeostasis and are crucial in transposon silencing during the development of the embryo. Besides cir-miRNAs highly stable in blood, piRNAs are also reported to be stably expressed in circulation. Interestingly, specific piRNAs have been useful in distinguishing between tumors and non-tumor tissues (piR-25447, piR-23992, piR-1043, piR-28876), and have been implicated in contributing to colorectal cancer development and risk (piR-019825, piR-015551). Nonetheless, identification and exploration piRNA that could aid in better classification of individuals and their symptom severities in ASD has not been previously undertaken. We found 22 piRNAs differentially and highly expressed in severely affected subjects' plasma while 7 were down-regulated. These piRNAs include piR-hsa-2813, the most up-regulated, and piR-hsa-27623, which was down-regulated. Thus, like the differentially expressed miRNA, these identified piRNAs can be used as biomarkers to aid in diagnosing ASD and stratifying between severe and mild ASD.

Deep sequencing platforms allow the identification of a considerable amount of noncoding RNA transcripts. In addition to miRNAs and piRNAs, recent analyses from high-throughput sequencing revealed the existence of other classes of ncRNAs, including snoRNAs and Y-RNAs, revealing a wide range of small regulatory RNAs with a wide variety of processing mechanisms and functions. Using small RNA high-throughput sequencing, we demonstrated that the ˜110 nucleotides (nt) long Ro-associated Y-RNAs (also called RNYs or Y-RNAs) are present in blood. We further found that Y-RNA, hY3, and pseudogene hY3P1 to be differentially down-regulated in severe cases. RNY4 pseudogene 28 and 29, were further identified to be differentially expressed in severe cases, down-regulated and up-regulated, respectively. Y-RNAs have emerged as playing a role in the initiation of chromosomal DNA replication, RNA stability, and cellular responses to stress. As with the other types of ncRNA, past investigations on Y-RNA have focused mainly on cancer research. However, accumulating evidence has shown that fragments of Y-RNAs displayed significant differential expression patterns both in circulation and/or in tumor tissues when compared to controls. While the particular functional significance of Y-RNA and its differential expression is less clear that for miRNA and piRNA, nonetheless Y-RNAs can also be used as biomarkers to aid in diagnosing ASD and stratifying between severe and mild ASD.

Similarly, snoRNAs are also differentially expressed. According to our analysis, the SNORA69 (known as U69) is the most up-regulated small nucleolar RNA, whereas SNORD42A (U42) is the most down-regulated snoRNA in individuals that expressed more severe symptoms of ASD. Interestingly, a microdeletion of a subtype of snoRNA (HBI-85), has been previously associated with Prader-Willi syndrome-like phenotypes. Prader-Willi syndrome has overlapping characteristics to ASD (e.g., social difficulties), lending credence to the idea that there is a pathophysiologic link between the differentially expressed snoRNAs and ASD symptomology. As with ncRNA above, snoRNAs can be used as biomarkers to aid in diagnosing ASD and stratifying between severe and mild ASD.

The herein disclosed data on differentially expressed ncRNA enables the construction of ncRNA expression profiles for severe or mild ASD. A more robust diagnosis is possible by assessing a plurality of ncRNA. While assessing all of the differentially expressed ncRNA would be unwieldly, panels can be assembled from subsets of the identified ncRNA, preferentially incorporating those providing the strongest signals. A panel can comprise a single biotype of ncRNA or multiple biotypes. In some instances a degree of technical ease can be obtained by restricting the biotype(s) used in a particular panel. For example, in some embodiments the RNA or cDNA can be size fractionated to enrich for certain biotypes (note that Y-RNA and snoRNA is substantially larger than miRNA or pi RNA). Thus in some embodiments, the panel comprises a single biotype: miRNA, piRNA, Y-RNA, or snoRNA. In other embodiments, the panel comprises multiple biotypes, for example miRNA and piRNA, or Y-RNA and snoRNA, etc. In various embodiments, the panel comprises at least 5-30 individual ncRNA (or any integer subrange or value therein). Exemplary panels comprising a single biotype of ncRNA are provided in Tables 1 and 2 (above). An exemplary panel comprising two biotypes of ncRNA is provided in Table 3 (above).

When using deep sequencing to assess an ncRNA profile, in some embodiments, a minimum number of reads per million (RPM) is assigned for each individual ncRNA. That is, the number sequence reads for the particular ncRNA are recorded per million total sequences read in the sample. In various embodiments, a single assessment may comprise at least 5, 10, 15, 20, 25, 30, 35, or 40 million reads per sample. For example, for various individual ncRNA to be considered to match the profile the level of expression can be >100 RPM, >200 RPM, >300 RPM, or <5 RPM, <10 RPM, <20 RPM. In some embodiments, all ncRNA in the panel must match the profile for a diagnosis or stratification to be made. In other embodiments, a diagnosis or stratification is made if ≥90% of the ncRNA in the panel match the profile.

### EXAMPLES

The following non-limiting examples are provided for illustrative purposes only in order to facilitate a more complete understanding of representative embodiments now contemplated. These examples should not be construed to limit any of the embodiments described in the present specification,

### Example 1

### Experimental Methods

Ethics statement. The Ministry of Public Health in Qatar has contributed respectable parameters to the local Institutional Review Board (IRB), with national guidelines that oversee research investigations comprised of vulnerable subjects such as children. These guidelines ensure the safety and wellbeing of these participants. Patient information was tightly controlled through limited access and password and data encrypted files. Furthermore, generated data is untraceable to ensure the confidentiality of participants. All participants were consented and informed about all aspects of the project. Moreover, all protocols, procedures, and subject/patient recruitment described in this study were conducted according to the principles expressed in the “Declaration of Helsinki” and approved by the ethical Institutional Review Board (IRB) committee of Qatar Biomedical Research Institute (QBRI-IRB:2018-024).

Subjects—The Interdisciplinary Research Program (IDRP) ASD cohort. Samples utilized in this study were obtained from a depository belonging to Qatar Biomedical Research Institute (QBRI) Interdisciplinary Research Program (IDRP) entitled Identifying Potential Molecular Biomarkers for Autism Spectrum Disorder. The umbrella study encompassed various disciplines and a blend of omic investigations to further our understanding of the fundamental underpinnings of Autism Spectrum Disorder and establish diagnostic tools for its early detection. Children ranging from the ages of 3-15 were recruited and their parents from within the Qatari population. ASD cases were subdivided based on those only had characteristic symptoms of ASD or were diagnosed to have ASD with associated comorbidity (i.e., attention-deficit/hyperactivity disorder (ADHD), intellectual disability (ID), or epilepsy). This study's strength will be in the varying attributes used to define the divisions within the cohort based on symptomatology and comorbidities. Age-matched control groups included siblings/healthy individuals from the general population and a neurodevelopmental disorder group of age-matched children that solely elicited ADHD, ID, or epilepsy. Consequently, the target cohort is to reach 600 ASD cases. For our current pilot study, we subdivided into those that exhibited severe ASD (n=22) and mild symptoms of ASD (n=23). The clinical characteristics of the subjects are described in Table 1.

ASD assessment. Children were clinically assessed and diagnosed with ASD at the Rumailah Hospital and Shaffalah Center for Children with Special Needs, Doha, Qatar. All children were diagnosed through a specialized, multidisciplinary team (MDT), consisting of medical doctors, psychiatrists, clinical nurse specialists, community mental health nurses, psychologists, social workers, and occupational therapists. Furthermore, validated screening and diagnostic tests and tools, including the Diagnostic and Statistical Manual of Mental disorders (DSM-V), Autism Diagnostic Observation Schedule, Second Edition (ADOS-2), and Autism Diagnostic Interview, Revised (ADI-R) were used.

Severity classification. Due to the complexity and heterogeneity of ASD, classifying an individual with the disorder is a perplexing endeavor. Hence, to respect and be sensitive to the extensive and multifaced classification of ASD diagnosis, we have divided our findings into two groups, the first of which represents individuals that exhibit severe symptoms displays multiple unambiguous characteristics of ASD, including severe behavioral phenotypes (i.e., significant alternations in social and language development), and those that show mild symptoms of ASD. To ensure that samples analyzed were grouped accordingly, ADOS-2 was used to verify the initial clinical diagnosis.

Collection of human blood/plasma. The collection of blood samples complied with the national guidelines that oversee research investigations comprising vulnerable subjects such as children. With extensive experience working with children with special needs, well-trained phlebotomists were responsible for collecting venous blood samples. Furthermore, using an EMLA cream for local anesthesia was incorporated to avoid and/or reduce pain sensitivity during blood withdrawal. Samples were collected into VACUETTE® tubes containing EDTA, centrifuged at 1800 rpm for 10 min, followed by plasma collection and re-centrifugation for 10 min at 3000 rpm. Finally, plasma samples were aliquoted into 200 μl aliquots and stored at −80° C. until further use.

RNA isolation from peripheral blood plasma. Frozen plasma samples were thawed in a 37° C. water bath. Thawed plasma samples were centrifuged at 400×g (˜2000 rpm) for 2 min to remove cells and precipitated plasma proteins/lipids. Cell-free (cf) plasma samples were transferred to new tubes for RNA isolation using miRNeasy Serum/Plasma Advanced Kit according to the manufacturer's instructions (Qiagen, Cat. no. 217204). We optimized the recommended starting amount of plasma; due to the low quantity of cfRNA, we used 200 μl of plasma for total RNA extraction with the addition of 52 QIAseq miRNA Library QC Spike-ins (Qiagen, Cat. no.: 331541) as an internal control for miRNA expression profiling in plasma.

QIAseq miRNA Library Quality Check. The QIAseq miRNA Library QC qPCR Assay Kit (Qiagen, Cat. no. 331551) was used to evaluate RNA isolation quality before small RNA library preparation and assess NGS performance post-sequencing. The kit provides 52 Spike-Ins controls with a qPCR panel that monitors the technical quality of the whole process from RNA isolation (by evaluating the reproducibility) to sequencing data analysis (by checking the reads). This method also enables detecting enzymatic inhibitors or nucleases and hemolysis assessment (necessary for plasma miRNA identification). Briefly, the procedure started during RNA isolation with the addition of 52 QIAseq miRNA Library QC Spike-Ins to the samples. The sample evaluation is determined using qRT-PCR. For the identification of RNA isolation efficiency, calculation of delta CT for UniSp100 (CT: 31-34 range) and UniSp101 (CT: 25-28 range) is assessed, and it should be around 5-7. For inhibitor detection, the UniSp6 is measured. The value should be <2 CTs between any two samples. For hemolysis, delta CT (miR-23a - miR-451a) should be less than 5 for high-quality samples. A value of 5-7 was considered a borderline sample. Samples with a value >7 were not be used.

Small RNA library preparation. For the library construction and molecular indexing, the QIAseq miRNA Library Kit (96) (Qiagen, Cat. no. 331505) and QIAseq miRNA NGS 96 Index IL (Qiagen, Cat. no. 331565) were used. The gold standard approach for normalization of circulating miRNAs utilizes equal amounts of biofluids and isolated total RNA and the spike-ins normalization controls. Thus, 5 μl of total RNA of 15 μl total RNA column eluate was used for library preparation. RNA samples were subjected to 3′ and 5′ adapter ligation targeting miRNAs by reverse transcription for generating the cDNA construct based on small RNA having 3′ and 5′ adapter ligation. This reverse transcription step will help enrich the RNA fragments with 3′ and 5′ adapters on both ends. The reverse transcription (RT) primer contained an integrated UMI (Unique Molecular Indices). The RT primer binds to a region of the 3′ adapter and facilitates converting the 3′/5′ ligated miRNAs into cDNA while assigning a UMI to every miRNA molecule. During reverse transcription, a universal sequence is also added. The sample indexing primers recognize that during library amplification. cDNA constructs were purified using a streamlined magnetic bead-based method. Then, unbiased amplification of libraries was accomplished using a dried universal forward primer from a plate paired with 1 of 96 dried reverse primers in the same plate (Qiagen, Cat. no. 331565).

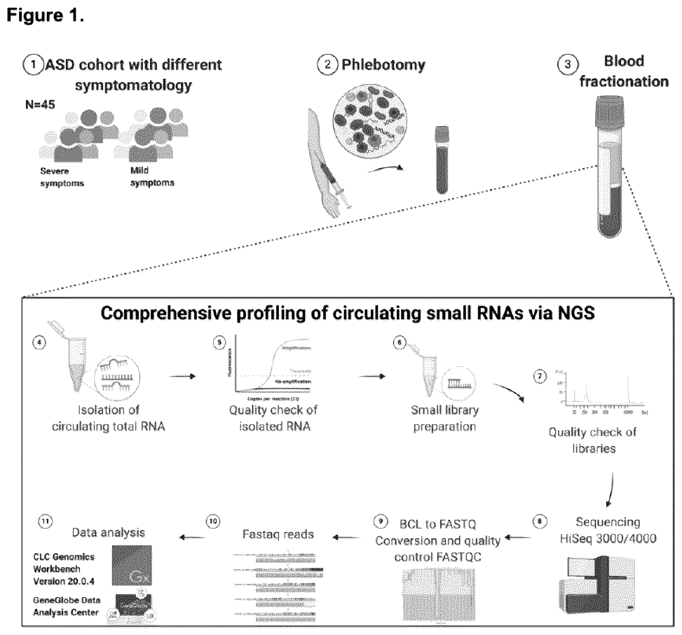

Consequently, this assigned each sample a unique custom index. After the library amplification, a cleanup was performed using the streamlined magnetic bead-based method again. Validation of the libraries was performed using Agilent technologies 2100 Bioanalyzer with an Agilent High Sensitivity DNA assay (Agilent, Cat. no. G2938-90020). A unique peak of around 141 bp was obtained (a purified library example is shown in FIG. 1).

Small RNA deep sequencing. cDNA libraries were measured based on the average size obtained from the bioanalyzer and by using Qubit Fluorometer, Qubit HS dsDNA Assay Kit (Life Technologies, Cat. no. Q32854). Libraries were diluted to 10 nM using a resuspension buffer and pooled with unique indexing for Illumina. The final dilution loaded was 3 nM, with further clustering on cBot2 performed, and sequencing on the Illumina platform achieved using the HiSeq 3000/4000 SBS Kit (150 cycles). For discovering novel miRNAs, we aimed to generate up to 20 million reads per sample. The adapters were trimmed. The raw data from the Illumina HiSeq 3000/4000 were converted from bcl2 to fastq format.

Sequencing read mapping and small RNA annotation. The raw sequence files from the Illumina HiSeq 3000/4000 in the form of BCL format were converted to the FASTQ format using the bcl2fastq v1.8.4 conversion tool. Reads were filtered, and adapters were trimmed. After adapter trimming, the read data was evaluated for quality using FASTQC to filter out reads with a quality score (Andrews, 2010 FastQC: a quality control tool for high throughput sequence data. Babraham Institute. https://www.bioinformatics.babraham.ac.uk/projects/fastqc/).

UMI (Unique Molecular Indices) analysis: The GeneGlobe data analysis center. The GeneGlobe data analysis enter (https://www.qiagen.com/us/shop/genes-and-pathways/data-analysis-center-overview-page/) can align and report on the QIAseq miRNA spike-ins in addition to the aligned small/miRNA/piRNA from each sample. This QIAGEN's analysis tool was used for assessing the effectiveness of QIAseq's UMIs. For the synthetic miRNA samples, the option ‘other’ was chosen for mapping, while ‘human’ was chosen for the human total RNA samples during the primary data analysis. The resulting count table included UMI and raw read counts for each miRNA in the samples. Before analyzing the correlation between UMI and raw read counts, the counts were rlog transformed.

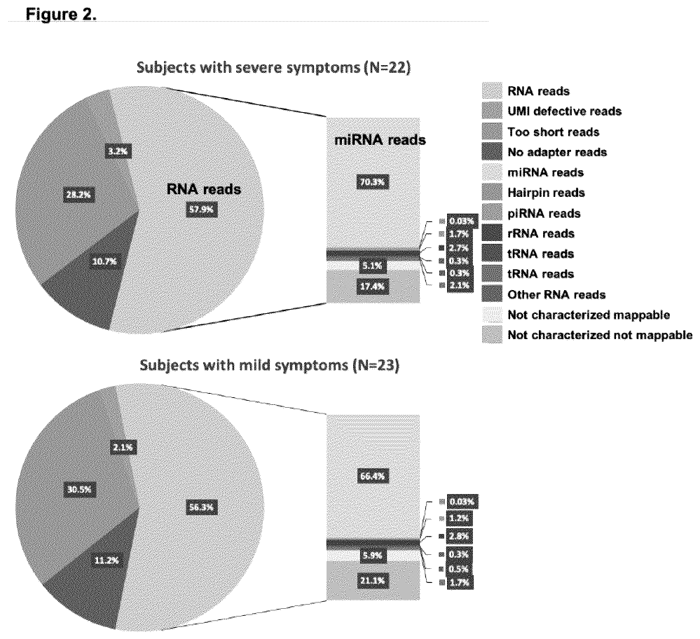

Next-generation sequencing (NGS) allows not only the quantification of known miRNAs but also the identification and quantification of novel miRNAs, isomiRs (miRNA variants), and other small RNA species that can be functionally relevant in diseases and therefore used as potential disease biomarker (FIG. 2). miRNAs are identified by aligning the reads to miRBase (version 21), and the reads are tallied to generate total counts for each miRNA. Statistical significance (p-value) between 2 or more samples were calculated to generate differential expression profiles.

Differential expression analysis: CLC Genomics Workbench version 20.0.4. Files were then exported to the CLC Genomics Workbench (version 20.0.4) for read mapping to the hg38 human genome version. This allowed for a single-mismatched base down to 18 nucleotides. Analysis of the resulting data was performed using small RNA analysis tools in CLC Genomics Workbench. Spike-in reads were filtered out from the rest of the data. “Perfect match” settings were applied when mapping, filtering, and counting QIAaseq NGS Spike-in reads in a dataset. Following counting of the QIAseq NGS Spike-in reads, they should be normalized to the total number of reads per sample. After this normalization, correlation matrices should be plotted for all sample-to-sample comparisons. This is done to evaluate the sample-to-sample correlation in the sample set. The expected correlation should be R2 of 0.95-0.99. If samples deviate from these values, they could be technical outliers and potentially be excluded from downstream analysis.

Using the Biomedical Genomics Analysis plugin that supports the analysis of reads sequenced using the QIAseq miRNA Library Kit, the QIAGEN miRNA Quantification workflow quantified the expression in each sample miRNAs found in miRBase. Reads were first mapped to databases of miRBase version 21 (http://www.mirbase.org) and piRNABank database Human_piRNA_sequence_v1.0 (http://www.regulatoryrna.org/database/piRNA/) to assign reads to miRNAs and piRNAs, respectively, and to exclude them before mapping to the full human genome. The unmapped reads from the QIAseq miRNA quantification workflow were collected and mapped using RNA-seq analysis to assign reads to other noncoding RNAs such as Y-RNAs and snoRNAs.

The QIAseq miRNA Quantification tool allows grouping of miRNA either as mature miRNA, the same mature miRNA may be produced from different precursor miRNAs, or on seed, the same seed sequence may be found in different mature miRNAs. A custom database for piRNAs was n seed was used for further analysis through the Ingenuity Pathway Analysis (IPA) platform. The workflow calculates differential expressions for expression tables with associated metadata using multi-factorial statistics based on a negative binomial Generalized Linear Model (GLM). Both Grouped on Mature and Grouped on Seed expression tables can be used. Integrated Unique Molecular Indices enable quantification of individual miRNA molecules, eliminating PCR and sequencing bias. For the differential expression analysis, miRNAs were deemed statistically differentially expressed if they had an expression of greater than 50 read counts at an absolute fold change >two and an adjusted P<0.05.

Functional enrichment tests. We used the Ingenuity Pathway Analysis (IPA) system for pathway analysis and molecular networks to perform the candidate miRNAs' functional enrichment tests. The IPA system provides a more comprehensive pathway resource based on manual collection. The rich information returned by IPA is also suitable for pathway crosstalk analysis, as it has almost all molecules with their connections included. Briefly, the IPA system implements Fisher's exact test to determine the pathways enriched with miRNAs of interest. Furthermore, the IPA system's network analysis searches for significant molecular networks in a commercial knowledge base, including integrative information from literature, gene expression, and gene annotation.

Patient characteristics and the design of the study. Our study analyzed a total of 45 children with ASD; 22 children with severe symptoms and 23 with mild symptoms. All subjects included in the study were assessed using either a multidisciplinary clinical assessment or DSM-V clinical diagnoses or a combined DSM-V and ADOS. Clinical details of the ASD cohort are summarized in Table 4. FIG. 1 illustrates the workflow that was followed in this study.

TABLE 4
Summary of clinical details fo the AD cohort.

| | | Severe | Mild |
|---|---|---|---|
| No. of cases | | 22 | 23 |
| Gender | Male | 16 (76%) | 18 (78%) |
| | Female | 7 (24%) | 5 (22%) |
| Age | Median ± SD | 7.6 ± 1.9 | 7.6 ± 2 |

### Example 2

Sequencing the Circulating Transcriptome of ASD Cases with Mild and Severe Symptoms.

Before library preparation and after RNA isolation, the expression levels of 5 miRNAs (miR-103, miR-191, miR-30c, miR-451 and miR-23) and 3 out of the 52 added spike-ins were evaluated based on qRT-PCR Ct values (Table 5). Unique spike-ins and qPCR-based miRNA quality control are crucial for low-abundance RNA samples. As described in the methods section, calculating delta CT for UniSp100 and UniSp101 enables distinguishing of outlier samples. The delta CT for the two spike-ins ranged between 5-7. UniSp6 evaluates the cDNA synthesis. The value should be <2 CTs between any two samples. Furthermore, it is crucial to evaluate hemolysis in plasma biomarker identification studies; in this case, the delta CT (miR-23a - miR-451a) was less than 5, indicating high-quality RNA samples. Endogenous miRNAs in plasma (miR-103, miR-191, and miR-30c) were also detected in all samples.

TABLE 5
Assays included for measuring spike-ins prior to library preparation.

| | | Results of | |
|---|---|---|---|
| Assay Quality Check Prior to NGS | Spike-Ins | Ct Average | Expected Results |
| RNA isolation efficiency | UniSp100 | 32 | Ct UniSp100-UniSp101 = 5-7 |
| RNA isolation efficiency | UniSp101 | 26 | |
| Endogenous control in plasma | miR-103 | 22 | Ct < 40 |
| Endogenous control in plasma | miR-191 | 18 | Ct < 40 |
| Hemolysis indicator in plasma | miR-30c | 24 | Ct < 40 |
| Hemolysis indicator in plasma | miR-451 | 25 | Ct miR-23a-miR-451a < 7 |
| Hemolysis indicator in plasma | miR-23 | 21 | |
| Monitoring presence of inhibitory | UniSp6 | 18 | <2 CTs between any two |
| components | | | samples |

Using Qiaseq library preparation and sequencing protocol, we sequenced cell-free RNA present in the plasma of ASD cases with severe and mild symptoms. Library construction was optimized using different starting amounts of plasma for RNA extraction. We found that doubling the starting recommended amount of plasma used for total RNA extraction (200 μl to 400 μl) improved libraries' quality.

The QIAseq miRNA sequencing data were analyzed first to the Qiagen GeneGlobe® Data Analysis Center, and the reads were processed as follows; for each sample, 20-30 million reads were obtained, more than 55% of reads were mapped to the human genome (hg19), and approximately 70% of these sequences were considered small RNA (sRNA), representing sequences between 18-43 nt (FIG. 2). All reads assigned to a particular miRNA or piRNA ID were counted, and the associated UMIs aggregated to count unique molecules. The largest category by frequency of reads was miRNAs, accounting for an average of 39.1% of reads (range 37.4-40.7%; FIG. 2). Read counts and UMI counts were presented in the output Excel® file “miR_piRNA” sheet. For sequences aligned with tRNAs or other RNAs, these results were displayed in the “tRNA” or “otherRNA” sheet, respectively. For sequences aligned to the genome at the last alignment step (this is performed for human using the most recent genome version), the same information (read counts and clustered UMIs) were output to the “notCharacterized_mappable” sheet. Remaining reads were also tallied (notCharacterized_notMappable) (FIG. 2).

miRNA expression analysis. The Biomedical Genomics Analysis plugin in the CLC Genomics Workbench software was used to quantify expression in each miRNA sample that was annotated and submitted to miRBase. Around 792 different human miRNA sequences were found in the samples, which accounted for approximately 1×106 and 10×106 reads for each sample. The top 20 miRNAs, consisting of >70% of mapped miRNAs reads, were well-known plasma abundant miRNAs; hsa-miR-16, hsa-miR-92a, has-miR-486-5p, hsa-miR-223, has-miR-122, members of the let-7 family (Table 6).

TABLE 6
Average of the most expressed 20 miRNAs found in the samples.

| Mature miRNA | Average |
|---|---|
| hsa-miR-16-5p | 29.5% |
| hsa-let-7b-5p | 14.6% |
| hsa-let-7a-5p | 8.4% |
| hsa-miR-486-5p | 6.6% |
| hsa-miR-122-5p | 4.2% |
| hsa-let-7f-5p | 4.1% |
| hsa-let-7i-5p | 2.6% |
| hsa-miR-223-3p | 2.2% |
| hsa-miR-142-3p | 2.2% |
| hsa-miR-451a | 2.1% |
| hsa-miR-92a-3p | 2% |
| hsa-miR-21-5p | 1.8% |
| hsa-miR-423-5p | 1.6% |
| hsa-miR-126-3p | 1.4% |
| hsa-miR-26b-5p | 1% |
| hsa-miR-26a-5p | 0.8% |
| hsa-miR-148a-3p | 0.7% |
| hsa-miR-25-3p | 0.6% |
| hsa-miR-101-3p | 0.5% |
| hsa-let-7g-5p | 0.4% |

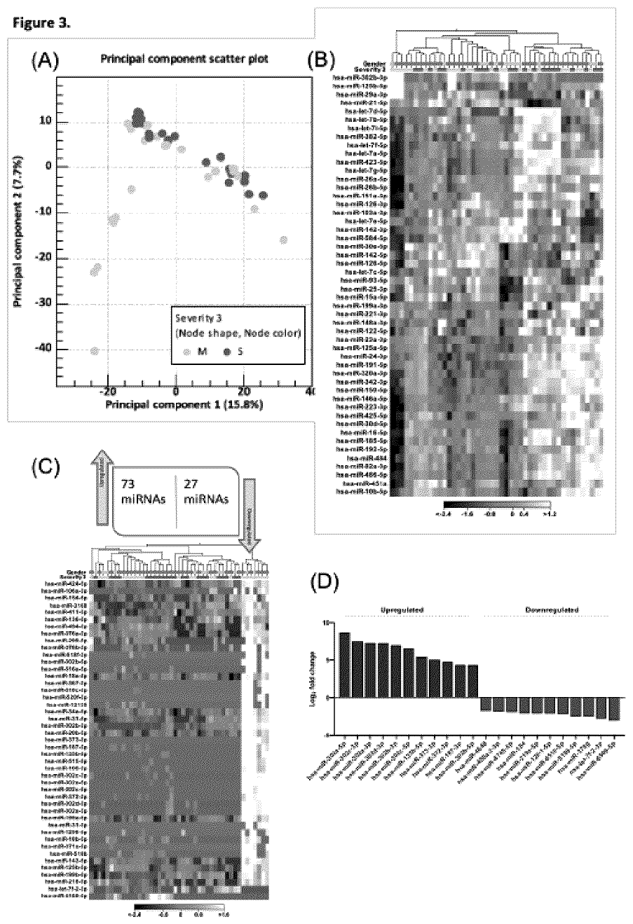

The analysis was performed by the CLC Genomic Workbench software using the QIAseq miRNA Differential Expression analysis with slightly modified settings that included a threshold to discard low background level intensities. Initially, a global view of gene expression profile through the Principal Component Analysis (PCA) between subjects that manifested severe symptoms of ASD (purple dots), and mild symptoms of ASD (yellow dots) samples was shown. PCA percent mapping on the top of the plot indicates the explained variability on the first coordinates (FIG. 3A). Then a two-dimensional heat map of expression values showed a hierarchical clustering analysis of miRNA expressed in both groups (FIG. 3B). The analysis allowed the identification of one hundred miRNAs differentially expressed between the different symptomatology of ASD (when using cutoff absolute fold change >2, p-value <0.05, >10 reads per sample; FIG. 3C). Seventy-three miRNAs were identified as being differentially expressed between the groups with higher expression levels in severe cases (fold change >2; p<0.05) (Table 6). Whereas twenty-seven miRNA showed significantly lower levels in the severe group compared to the mild (fold change <2; p<0.05) (Table 8).

We observed that the miRNA-302 family (hsa-miR-302a-5p, hsa-miR-302c-3p, hsa-miR-302a-3p, hsa-miR-302d-3p, hsa-miR-302b-3p, hsa-miR-302c-5p and hsa-miR-302b-5p) were expressed at significantly high levels in individuals that expressed severe characteristics of ASD in comparison to those that were mild. Previous findings have shown that miR-302 family is crucial in stem cell pluripotency and renewal and somatic cell DNA demethylation. Moreover, we found miR-135b-5p was expressed at high levels in severe cases vs. mild. It has been previously described that variable regulation of DISC1 by miR-135b-5p in the brain may prompt neuropsychiatric phenotypes.

TABLE 7
Differentially expressed miRNAs (N = 73; abs fold change > 2; p < 0.05)
Increased expression in severe ASD as compared to mild.

| | | Log2 | | | |
|---|---|---|---|---|---|
| | | fold | Fold | | |
| # | ID | change | change | p-value | Symbol |
| 1 | hsa- | 8.70 | 416.11 | 2.36E−14 | miR-302a-5p (miRNAs w/seed CUUAAAC; |
| | miR- | | | | SEQ ID NO: 1) |
| | 302a- | | | | |
| | 5p | | | | |
| 2 | hsa- | 7.51 | 182.42 | 4.6E−14 | miR-291a-3p (and other miRNAs w/seed |
| | miR- | | | | AAGUGCU; SEQ ID NO: 2) |
| | 302c- | | | | |
| | 3p | | | | |
| 3 | hsa- | 7.24 | 152.01 | 4.85E−14 | miR-291a-3p (and other miRNAs w/seed |
| | miR- | | | | AAGUGCU; SEQ ID NO: 2) |
| | 302a- | | | | |
| | 3p | | | | |
| 4 | hsa- | 7.23 | 150.37 | 8.33E−15 | miR-291a-3p (and other miRNAs w/seed |
| | miR- | | | | AAGUGCU; SEQ ID NO: 2) |
| | 302d- | | | | |
| | 3p | | | | |
| 5 | hsa- | 6.95 | 123.89 | 2.22E−16 | miR-291a-3p (and other miRNAs w/seed |
| | miR- | | | | AAGUGCU; SEQ ID NO: 2) |
| | 302b- | | | | |
| | 3p | | | | |
| 6 | hsa- | 6.56 | 94.36 | 1.47E−09 | miR-302c-5p (miRNAs w/seed UUAACAU; |
| | miR- | | | | SEQ ID NO: 3) |
| | 302c- | | | | |
| | 5p | | | | |
| 7 | hsa- | 5.49 | 44.93 | 2.2E−07 | miR-135a-5p (and other miRNAs w/seed |
| | miR- | | | | AUGGCUU; SEQ ID NO: 4) |
| | 135b- | | | | |
| | 5p | | | | |
| 8 | hsa- | 5.02 | 32.39 | 1.76E−05 | miR-291a-3p (and other miRNAs w/seed |
| | miR- | | | | AAGUGCU; SEQ ID NO: 2) |
| | 373-3p | | | | |
| 9 | hsa- | 4.75 | 26.90 | 8.48E−07 | miR-291a-3p (and other miRNAs w/seed |
| | miR- | | | | AAGUGCU; SEQ ID NO: 2) |
| | 372-3p | | | | |
| 10 | hsa- | 4.37 | 20.66 | 6.04E−06 | miR-187-3p (miRNAs w/seed CGUGUCU; |
| | miR- | | | | SEQ ID NO: 5) |
| | 187-3p | | | | |
| 11 | hsa- | 4.36 | 20.48 | 0.0001 | miR-302b-5p (and other miRNAs w/seed |
| | miR- | | | | CUUUAAC; SEQ ID NO: 6) |
| | 302b- | | | | |
| | 5p | | | | |
| 12 | hsa- | 4.32 | 19.96 | 1.26E−05 | miR-100-3p (miRNAs w/seed AAGCUUG; |
| | miR- | | | | SEQ ID NO: 7) |
| | 100-3p | | | | |
| 13 | hsa- | 4.31 | 19.82 | 6.95E−05 | miR-12135 (miRNAs w/seed AAAGGUU; |
| | miR- | | | | SEQ ID NO: 8) |
| | 12135 | | | | |
| 14 | hsa- | 4.29 | 19.51 | 9.49E−05 | miR-293-5p (and other miRNAs w/seed |
| | miR- | | | | CUCAAAC; SEQ ID NO: 9) |
| | 371a- | | | | |
| | 5p | | | | |
| 15 | hsa- | 4.25 | 18.99 | 0.0001 | miR-518a-3p (and other miRNAs w/seed |
| | miR- | | | | AAAGCGC; SEQ ID NO: 10) |
| | 518c- | | | | |
| | 3p | | | | |
| 16 | hsa- | 4.05 | 16.57 | 7.08E−05 | miR-515-5p (and other miRNAs w/seed |
| | miR- | | | | UCUCCAA; SEQ ID NO: 11) |
| | 515-5p | | | | |
| 17 | hsa- | 3.98 | 15.73 | 0.0004 | miR-31-3p (and other miRNAs w/seed |
| | miR- | | | | GCUAUGC; SEQ ID NO: 12) |
| | 31-3p | | | | |
| 18 | hsa- | 3.85 | 14.39 | 0.0007 | miR-302c-3p (and other miRNAs w/seed |
| | miR- | | | | AGUGCUU; SEQ ID NO: 13) |
| | 520f-3p | | | | |
| 19 | hsa- | 3.66 | 12.67 | 0.0001 | miR-516a-5p (miRNAs w/seed UCUCGAG; |
| | miR- | | | | SEQ ID NO: 14) |
| | 516a- | | | | |
| | 5p | | | | |
| 20 | hsa- | 3.49 | 11.26 | 0.0014 | miR-518a-3p (and other miRNAs w/seed |
| | miR- | | | | AAAGCGC; SEQ ID NO: 10) |
| | 518f-3p | | | | |
| 21 | hsa- | 3.48 | 11.18 | 0.0019 | miR-2682-3p (and other miRNAs w/seed |
| | miR- | | | | GCCUCUU; SEQ ID NO: 15) |
| | 6781- | | | | |
| | 3p | | | | |
| 22 | hsa- | 3.32 | 10.02 | 0.0009 | miR-1298-5p (and other miRNAs w/seed |
| | miR- | | | | UCAUUCG; SEQ ID NO: 16) |
| | 1298- | | | | |
| | 5p | | | | |
| 23 | hsa- | 3.32 | 9.98 | 7.01E−09 | miR-31-5p (and other miRNAs w/seed |
| | miR- | | | | GGCAAGA; SEQ ID NO: 17) |
| | 31-5p | | | | |
| 24 | hsa- | 3.30 | 9.86 | 1.47E−05 | miR-376a-3p (and other miRNAs w/seed |
| | miR- | | | | UCAUAGA; SEQ ID NO: 18) |
| | 376b- | | | | |
| | 3p | | | | |
| 25 | hsa- | 3.26 | 9.58 | 0.0004 | miR-106a-3p (and other miRNAs w/seed |
| | miR- | | | | UGCAAUG; SEQ ID NO: 19) |
| | 106a- | | | | |
| | 3p | | | | |
| 26 | hsa- | 3.16 | 8.93 | 0.0030 | miR-376b-5p (and other miRNAs w/seed |
| | miR- | | | | GUGGAUA; SEQ ID NO: 20) |
| | 376c- | | | | |
| | 5p | | | | |
| 27 | hsa- | 3.07 | 8.41 | 0.0047 | miR-517a-3p (and other miRNAs w/seed |
| | miR- | | | | UCGUGCA; SEQ ID NO: 21) |
| | 517c- | | | | |
| | 3p | | | | |
| 28 | hsa- | 2.89 | 7.39 | 0.0023 | miR-517a-3p (and other miRNAs w/seed |
| | miR- | | | | UCGUGCA; SEQ ID NO: 21) |
| | 517a- | | | | |
| | 3p | | | | |
| 29 | hsa- | 2.74 | 6.68 | 0.0012 | miR-518a-3p (and other miRNAs w/seed |
| | miR- | | | | AAAGCGC; SEQ ID NO: 10) |
| | 518b | | | | |
| 30 | hsa- | 2.67 | 6.38 | 0.0009 | miR-18a-5p (and other miRNAs w/seed |
| | miR- | | | | AAGGUGC; SEQ ID NO: 22) |
| | 18b-5p | | | | |
| 31 | hsa- | 2.57 | 5.95 | 0.0193 | miR-520d-5p (and other miRNAs w/seed |
| | miR- | | | | UACAAAG; SEQ ID NO: 23) |
| | 524-5p | | | | |
| 32 | hsa- | 2.545 | 5.84 | 0.0203 | miR-4639-5p (miRNAs w/seed UGCUAAG; |
| | miR- | | | | SEQ ID NO: 23) |
| | 4639- | | | | |
| | 5p | | | | |
| 33 | hsa- | 2.41 | 5.31 | 0.0041 | miR-376b-5p (and other miRNAs w/seed |
| | miR- | | | | GUGGAUA; SEQ ID NO: 20) |
| | 376b- | | | | |
| | 5p | | | | |
| 34 | hsa- | 2.28 | 4.86 | 0.0389 | miR-105-5p (and other miRNAs w/seed |
| | miR- | | | | CAAAUGC; SEQ ID NO: 24) |
| | 105-5p | | | | |
| 35 | hsa- | 2.27 | 4.81 | 0.0064 | miR-3195 (miRNAs w/seed GCGCCGG; |
| | miR- | | | | SEQ ID NO: 25) |
| | 3195 | | | | |
| 36 | hsa- | 2.27 | 4.81 | 0.0276 | miR-5480-3p (and other miRNAs w/seed |
| | miR- | | | | CAAAACU; SEQ ID NO: 26) |
| | 1323 | | | | |
| 37 | hsa- | 2.28 | 4.71 | 2.83E−07 | miR-376a-3p (and other miRNAs w/seed |
| | miR- | | | | UCAUAGA; SEQ ID NO: 18) |
| | 376a- | | | | |
| | 3p | | | | |
| 38 | hsa- | 2.22 | 4.66 | 0.00247 | miR-138-5p (miRNAs w/seed GCUGGUG; |
| | miR- | | | | SEQ ID NO: 27) |
| | 138-5p | | | | |
| 39 | hsa- | 2.19 | 4.57 | 2.68E−05 | miR-143-5p (and other miRNAs w/seed |
| | miR- | | | | GUGCAGU; SEQ ID NO: 28) |
| | 143-5p | | | | |
| 40 | hsa- | 2.19 | 4.56 | 4.06E−07 | miR-34a-5p (and other miRNAs w/seed |
| | miR- | | | | GGCAGUG; SEQ ID NO: 29) |
| | 34a-5p | | | | |
| 41 | hsa- | 2.16 | 4.47 | 0.0006 | miR-299a-5p (and other miRNAs w/seed |
| | miR- | | | | GGUUUAC; SEQ ID NO: 30) |
| | 299-5p | | | | |
| 42 | hsa- | 1.99 | 3.99 | 0.004 | miR-485-3p (and other miRNAs w/seed |
| | miR- | | | | UCAUACA; SEQ ID NO: 31) |
| | 539-3p | | | | |
| 43 | hsa- | 1.90 | 3.73 | 0.03 | miR-222-5p (miRNAs w/seed UCAGUAG; |
| | miR- | | | | SEQ ID NO: 32) |
| | 222-5p | | | | |
| 44 | hsa- | 1.86 | 3.63 | 0.001 | miR-3168 (miRNAs w/seed AGUUCUA; |
| | miR- | | | | SEQ ID NO: 33) |
| | 3168 | | | | |
| 45 | hsa- | 1.85 | 3.60 | 0.03 | miR-503-3p (miRNAs w/seed GGGUAUU; |
| | miR- | | | | SEQ ID NO: 34) |
| | 503-3p | | | | |
| 46 | hsa- | 1.85 | 3.59 | 0.001 | miR-154-5p (miRNAs w/seed AGGUUAU; |
| | miR- | | | | SEQ ID NO: 35) |
| | 154-5p | | | | |
| 47 | hsa- | 1.84 | 3.59 | 0.0001 | miR-136-5p (miRNAs w/seed CUCCAUU; |
| | miR- | | | | SEQ ID NO: 36) |
| | 136-5p | | | | |
| 48 | hsa- | 1.75 | 3.36 | 0.0003 | miR-218-5p (and other miRNAs w/seed |
| | miR- | | | | UGUGCUU; SEQ ID NO: 37) |
| | 218-5p | | | | |
| 49 | hsa- | 1.72 | 3.29 | 0.03 | miR-15a-3p (miRNAs w/seed AGGCCAU; |
| | miR- | | | | SEQ ID NO: 38) |
| | 15a-3p | | | | |
| 50 | hsa- | 1.70 | 3.26 | 0.03 | miR-1587 (and other miRNAs w/seed |
| | miR- | | | | UGGGCUG; SEQ ID NO: 39) |
| | 3620- | | | | |
| | 5p | | | | |
| 51 | hsa- | 1.70 | 3.26 | 0.0002 | miR-199a-5p (and other miRNAs w/seed |
| | miR- | | | | CCAGUGU; SEQ ID NO: 40) |
| | 199b- | | | | |
| | 5p | | | | |
| 52 | hsa- | 1.67 | 3.17 | 0.04 | miR-363-5p (and other miRNAs w/seed |
| | miR- | | | | GGGUGGA; SEQ ID NO: 41) |
| | 363-5p | | | | |
| 53 | hsa- | 1.56 | 2.95 | 0.002 | miR-122b-3p (and other miRNAs w/seed |
| | miR- | | | | AACACCA; SEQ ID NO: 42) |
| | 21-3p | | | | |
| 54 | hsa- | 1.48 | 2.79 | 1.74E−05 | miR-17-5p (and other miRNAs w/seed |
| | miR- | | | | AAAGUGC; SEQ ID NO: 43) |
| | 20b-5p | | | | |
| 55 | hsa- | 1.46 | 2.76 | 3.73E−05 | miR-16-5p (and other miRNAs w/seed |
| | miR- | | | | AGCAGCA; SEQ ID NO: 44) |
| | 424-5p | | | | |
| 56 | hsa- | 1.48 | 2.73 | 2.53E−05 | miR-125b-5p (and other miRNAs w/seed |
| | miR- | | | | CCCUGAG; SEQ ID NO: 45) |
| | 125b- | | | | |
| | 5p | | | | |
| 57 | hsa- | 1.42 | 2.68 | 0.04 | miR-135a-5p (and other miRNAs w/seed |
| | miR- | | | | AUGGCUU; SEQ ID NO: 4) |
| | 135a- | | | | |
| | 5p | | | | |
| 58 | hsa- | 1.37 | 2.59 | 0.001 | miR-494-3p (miRNAs w/seed GAAACAU; |
| | miR- | | | | SEQ ID NO: 46) |
| | 494-3p | | | | |
| 59 | hsa- | 1.37 | 2.58 | 0.03 | miR-411-3p (and other miRNAs w/seed |
| | miR- | | | | AUGUAAC; SEQ ID NO: 47) |
| | 379-3p | | | | |
| 60 | hsa- | 1.37 | 2.58 | 0.0135 | miR-125b-1-3p (miRNAs w/seed |
| | miR- | | | | CGGGUUA; SEQ ID NO: 48) |
| | 125b- | | | | |
| | 1-3p | | | | |
| 61 | hsa- | 1.32 | 2.50 | 3.06E−05 | miR-199a-5p (and other miRNAs w/seed |
| | miR- | | | | CCAGUGU; SEQ ID NO: 40) |
| | 199a- | | | | |
| | 5p | | | | |
| 62 | hsa- | 1.32 | 2.49 | 0.002 | miR-455-5p (and other miRNAs w/seed |
| | miR- | | | | AUGUGCC; SEQ ID NO: 49) |
| | 455-5p | | | | |
| 63 | hsa- | 1.31 | 2.48 | 0.001 | miR-411-5p (and other miRNAs w/seed |
| | miR- | | | | AGUAGAC; SEQ ID NO: 50) |
| | 411-5p | | | | |
| 64 | hsa- | 1.27 | 2.41 | 0.002 | miR-100-5p (and other miRNAs w/seed |
| | miR- | | | | ACCCGUA; SEQ ID NO: 51) |
| | 100-5p | | | | |
| 65 | hsa- | 1.26 | 2.39 | 0.006 | miR-542-3p (miRNAs w/seed GUGACAG; |
| | miR- | | | | SEQ ID NO: 52) |
| | 542-3p | | | | |
| 66 | hsa- | 1.23 | 2.34 | 0.008 | miR-377-3p (miRNAs w/seed UCACACA; |
| | miR- | | | | SEQ ID NO: 53) |
| | 377-3p | | | | |
| 67 | hsa- | 1.15 | 2.22 | 0.0002 | miR-18a-5p (and other miRNAs w/seed |
| | miR- | | | | AAGGUGC; SEQ ID NO: 22) |
| | 18a-5p | | | | |
| 68 | hsa- | 1.14 | 2.21 | 0.003 | miR-493-3p (miRNAs w/seed GAAGGUC; |
| | miR- | | | | SEQ ID NO: 54) |
| | 493-3p | | | | |
| 69 | hsa- | 1.10 | 2.15 | 0.019 | miR-1277-5p (miRNAs w/seed AAUAUAU; |
| | miR- | | | | SEQ ID NO: 55) |
| | 1277- | | | | |
| | 5p | | | | |
| 70 | hsa- | 1.04 | 2.05 | 0.024 | miR-136-3p (miRNAs w/seed AUCAUCG; |
| | miR- | | | | SEQ ID NO: 56) |
| | 136-3p | | | | |
| 71 | hsa- | 1.03 | 2.04 | 0.043 | miR-149-5p (miRNAs w/seed CUGGCUC; |
| | miR- | | | | SEQ ID NO: 57) |
| | 149-5p | | | | |
| 72 | hsa- | 1.02 | 2.02 | 0.042 | miR-145-3p (miRNAs w/seed GAUUCCU; |
| | miR- | | | | SEQ ID NO: 58) |
| | 145-3p | | | | |
| 73 | hsa- | 1.01 | 2.01 | 0.002 | miR-145-5p (and other miRNAs w/seed |
| | miR- | | | | UCCAGUU; SEQ ID NO: 59) |
| | 145-5p | | | | |

TABLE 8
Differentially expressed miRNAs (N = 27; fold change < 2; p < 0.05).
Decreased expression in severe ASD as compared to mild.

| | | Log2 | | | |
|---|---|---|---|---|---|
| | | fold | Fold | | |
| # | ID | change | change | p-value | Symbol |
| 1 | hsa-miR- | −1.04 | −2.06 | 0.016 | miR-3104-5p (and other miRNAs w/seed |
| | 6805-5p | | | | AGGGGGC; SEQ ID NO: 60) |
| 2 | hsa-miR- | −1.18 | −2.26 | 0.008 | miR-1-3p (and other miRNAs w/seed |
| | 206 | | | | GGAAUGU; SEQ ID NO: 61) |
| 3 | hsa-miR- | −1.21 | −2.31 | 0.018 | miR-197-5p (and other miRNAs w/seed |
| | 197-5p | | | | GGGUAGA; SEQ ID NO: 62) |
| 4 | hsa-miR- | −1.24 | −2.37 | 0.049 | miR-1247-3p (and other miRNAs w/seed |
| | 1292-5p | | | | GGGAACG; SEQ ID NO: 63) |
| 5 | hsa-miR- | −1.26 | −2.39 | 0.038 | miR-5481 (miRNAs w/seed AAAGUAU; |
| | 5481 | | | | SEQ ID NO: 64) |
| 6 | hsa-miR- | −1.31 | −2.49 | 0.023 | miR-181a-5p (and other miRNAs w/seed |
| | 181c-5p | | | | ACAUUCA; SEQ ID NO: 65) |
| 7 | hsa-miR- | −1.40 | −2.64 | 0.028 | miR-412-5p (miRNAs w/seed GGUCGAC; |
| | 412-5p | | | | SEQ ID NO: 66) |
| 8 | hsa-miR- | −1.42 | −2.69 | 0.038 | miR-548h-5p (and other miRNAs w/seed |
| | 548b-5p | | | | AAAGUAA; SEQ ID NO: 67) |
| 9 | hsa-miR- | −1.47 | −2.77 | 0.021 | miR-3150b-3p (and other miRNAs w/seed |
| | 3150b-3p | | | | GAGGAGA; SEQ ID NO: 68) |
| 10 | hsa-miR- | −1.50 | −2.83 | 0.016 | miR-12118 (and other miRNAs w/seed |
| | 6891-5p | | | | AAGGAGG; SEQ ID NO: 69) |
| 11 | hsa-miR- | −1.53 | −2.93 | 0.032 | miR-151-5p (and other miRNAs w/seed |
| | 151b | | | | CGAGGAG; SEQ ID NO: 70) |
| 12 | hsa-miR- | −1.55 | −2.93 | 0.029 | miR-216a-5p (miRNAs w/seed |
| | 216a-5p | | | | AAUCUCA; SEQ ID NO: 71) |
| 13 | hsa-miR- | −1.59 | −3.02 | 0.025 | miR-202-5p (miRNAs w/seed UCCUAUG; |
| | 202-5p | | | | SEQ ID NO: 72) |
| 14 | hsa-miR- | −1.65 | −3.16 | 0.016 | miR-5480-3p (and other miRNAs w/seed |
| | 5480-3p | | | | CAAAACU; SEQ ID NO: 26) |
| 15 | hsa-miR- | −1.68 | −3.21 | 0.03 | miR-378a-3p (and other miRNAs w/seed |
| | 378d | | | | CUGGACU; SEQ ID NO: 73) |
| 16 | hsa-miR- | −1.77 | −3.42 | 0.038 | miR-149-3p (and other miRNAs w/seed |
| | 6785-5p | | | | GGGAGGG; SEQ ID NO: 74) |
| 17 | hsa-miR- | −1.79 | −3.47 | 0.044 | miR-4648 (miRNAs w/seed GUGGGAC; |
| | 4648 | | | | SEQ ID NO: 75) |
| 18 | hsa-miR- | −1.91 | −3.76 | 0.012 | miR-450b-3p (and other miRNAs w/seed |
| | 450a-2-3p | | | | UUGGGGA; SEQ ID NO: 76) |
| 19 | hsa-miR- | −1.95 | −3.85 | 0.049 | miR-4481 (and other miRNAs w/seed |
| | 4745-5p | | | | GAGUGGG; SEQ ID NO: 77) |
| 20 | hsa-miR- | −2.11 | −4.32 | 0.006 | miR-184 (and other miRNAs w/seed |
| | 184 | | | | GGACGGA; SEQ ID NO: 78) |
| 21 | hsa-miR- | −2.12 | −4.36 | 0.018 | miR-219a-5p (and other miRNAs w/seed |
| | 219a-5p | | | | GAUUGUC; SEQ ID NO: 79) |
| 22 | hsa-miR- | −2.13 | −4.39 | 0.019 | miR-128-1-5p (and other miRNAs w/seed |
| | 128-1-5p | | | | GGGGCCG; SEQ ID NO: 80) |
| 23 | hsa-miR- | −2.22 | −4.67 | 0.007 | miR-6516-5p (miRNAs w/seed |
| | 6516-5p | | | | UUGCAGU; SEQ ID NO: 81) |
| 24 | hsa-miR- | −2.52 | −5.74 | 0.001 | miR-1285-3p (and other miRNAs w/seed |
| | 5189-5p | | | | CUGGGCA; SEQ ID NO: 82) |
| 25 | hsa-miR- | −2.53 | −5.79 | 0.003 | miR-378g (miRNAs w/seed CUGGGCU; |
| | 378g | | | | SEQ ID NO: 83) |
| 26 | hsa-let- | −2.79 | −6.94 | 0.0002 | let-7f-2-3p (and other miRNAs w/seed |
| | 7f-2-3p | | | | UAUACAG; SEQ ID NO: 84) |
| 27 | hsa-miR- | −3.05 | −8.33 | 0.008 | miR-6509-5p (miRNAs w/seed |
| | 6509-5p | | | | UUAGGUA; SEQ ID NO: 85) |

### Example 3

### Pathway Enrichment by Ingenuity Pathway Analysis (IPA)

Further functional enrichment tests were performed using Ingenuity Pathway Analysis (IPA) for both pathway analysis and the dataset's molecular networks representing 100 miRNAs with altered expression profiles obtained from the CLC Genomic Workbench v20.0.4. These differentially expressed miRNAs were imported into the Ingenuity Pathway Analysis Tool, and the following data is shown in Table 9 and Table 10: a) The list of top five Diseases and Disorders, b) Molecular and Cellular Functions, c) Physiological System Development and Function, d) networks with their respective scores obtained from IPA. In general, therefore, it seems that two out of five of the “Diseases and Disorders” list are related to psychological and neurological disorders, supporting the neurology implication hypothesis of these miRNAs (Table 9).

TABLE 9
Ingenuity Pathways Analysis (IPA) summary.

| | | | # |
|---|---|---|---|
| | Name | p-value range | Molecules |
| Diseases and | Organismal Injury and | 4.92E−02-1.45E−25 | 50 |
| Disorders | Abnormalities | | |
| | Reproductive System Disease | 4.74E−02-1.45E−25 | 36 |
| | Cancer | 4.97E−02-9.43E−17 | 35 |
| | Psychological Disorders | 2.70E−03-3.09E−15 | 23 |
| | Neurological Disease | 4.72E−02-4.38E−15 | 28 |
| Molecular and | Cellular Movement | 4.04E−02-2.17E−06 | 18 |
| Cellular Functions | Cell Cycle | 3.66E−02-2.62E−11 | 9 |
| | Cellular Development | 4.72E−02-3.93E−11 | 23 |
| | Cellular Growth and Proliferation | 3.66E−02-3.48E−09 | 21 |
| | Cellular Response to | 4.41E−02-3.27E−03 | 4 |
| | Therapeutics | | |
| Physiologic System | Digestive System Development | 1.11E−05-1.11E−05 | 4 |
| Development and | and Function | | |
| Function | Hepatic System Development | 3.44E−02-8.24E−14 | 4 |
| | and Function | | |
| | Organ Development | 3.66E−02-1.20E−20 | 7 |
| | Embryonic Development | 2.32E−02-8.24E−14 | 9 |
| | Connective Tissue Development | 3.66E−02-8.24E−14 | 3 |
| | and Function | | |

TABLE 10
Ingenuity Pathways Analysis (IPA) networks.

| | | | Focus | Top Diseases |
|---|---|---|---|---|
| ID | Molecules in Network | Score | Molecules | and Functions |
| 1 | BRAF, ERBB2, let-7f-2-3p, miR-106a-3p, mir-1231, miR-1285- | 40 | 19 | [Cancer, |
| | 3p, mir-138, miR-138-5p, | | | Organismal |
| | miR-149-5p, miR-151-5p, mir-154, miR-154-5p, mir-17, mir- | | | Injury and |
| | 184, mir-187, miR-187-3p, miR- 18a-5p, mir-197, miR-197-5p, | | | Abnormalities, |
| | miR-216a-5p, mir-25, miR-291a-3p, mir-302, miR-302a-5p, | | | Reproductive |
| | miR-302b-5p, miR-302c-5p, miR-377-3p, miR-485-3p, miR- | | | System Disease] |
| | 494-3p, OIP5-AS1, PTEN, RNF149, SRC (family), UCA1 | | | |
| 2 | calcifediol, EGFR, HARS1, IGF1R, mir-10, miR-100-3p, miR- | 32 | 16 | [Cancer, |
| | 100-5p, miR-105-5p, miR- 125b-1-3p, mir-136, miR-136-3p, | | | Gastrointestinal |
| | miR-136-5p, miR-16-5p, mir-25, mir-299, miR-299a-5p, mir-31, | | | Disease, |
| | miR-31-3p, miR-31-5p, miR-3104-5p, mir-322, mir-368, miR- | | | Organismal |
| | 378a-3p, mir-379, miR- 411-3p, miR-411-5p, mir-542, miR- | | | Injury and |
| | 542-3p, miR-92a-3p, PAX3-FOXO1, PIK3C2B, PTPN7, | | | Abnormalities] |
| | RECK, RTL1, TLR2 | | | |
| 3 | AGO1, AGO2, ALOX5AP, ARGONAUTE, DDX20, FGF16, | 29 | 15 | [Gene |
| | MEF2A, miR-100-5p, mir-122, | | | Expression, |
| | miR-122b-3p, mir-135, miR-135a-5p, mir-143, miR-143-5p, mir- | | | Organismal |
| | 15, mir-154, miR-15a-3p, mir-199, miR-199a-5p, miR-21-5p, | | | Injury and |
| | mir-219, miR-219a-5p, miR-3150b-3p, mir-363, miR-363- 5p, | | | Abnormalities, |
| | mir-455, miR-455-5p, mir-493, miR-493-3p, miR-494-3p, miR- | | | Reproductive |
| | 515-5p, mir-548, miR-5480-3p, miR-92a-3p, ZBP1 | | | System Disease] |
| 4 | Akt, CDKN1C, GSS, HIPK3, Insulin, KCNJ16, miR-1-3p, miR- | 22 | 12 | [Cancer, |
| | 125b-5p, mir-128, miR-128- | | | Organismal |
| | 1-5p, miR-1298-5p, miR-17-5p, miR-181a-5p, miR-218-5p, | | | Injury and |
| | mir-25, mir-290, miR-293-5p, miR-34a-5p, mir-363, mir-368, | | | Abnormalities, |
| | miR-376a-3p, miR-376b-5p, miR-92a-3p, MYLIP, OIP5-AS1, | | | Reproductive |
| | OSBPL8, PREX2, PTK2, SLC18A2, SLC22A17, SLIT2, | | | System Disease] |
| | Smad2/3, SMAD6/7, THEMIS, VSNL1 | | | |
| 5 | ACTA2, AR, BCDIN3D, CACNA1C, CD46, CDK11B, CFL2, | 22 | 12 | [Cell Cycle, |
| | DKK1, MACC1-AS1, MED28, MICA, mir-145, miR-145-3p, | | | Organismal |
| | miR-145-5p, miR-149-3p, miR-222-5p, miR-291a-3p, mir-302, | | | Injury and |
| | miR-302c-3p, mir-515, miR-516a-5p, miR-517a-3p, miR-518a- | | | Abnormalities, |
| | 3p, miR-520d-5p, mir-548, | | | Reproductive |
| | miR-548h-5p, miR-548l, PBK, SMOOTH MUSCLE ACTIN, | | | System Disease] |
| | Snhg14, TMEM8B, TUG1, USP12, VPS26A, VSNL1 | | | |

### Example 4

### Molecular Networks

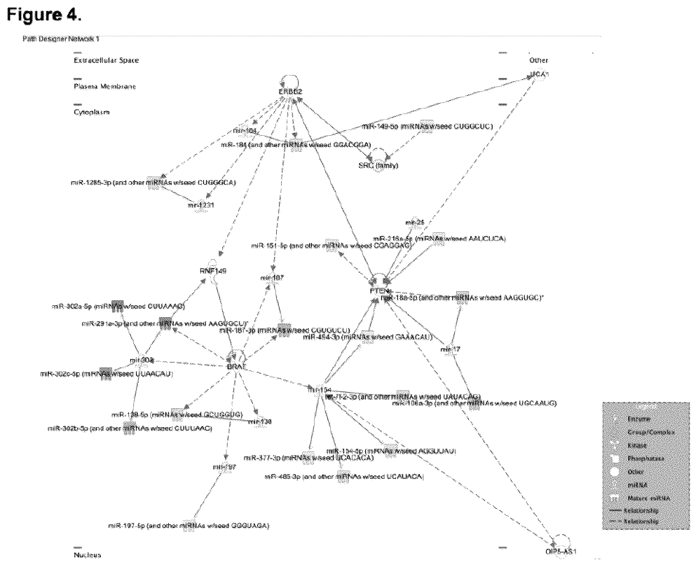

The network analysis in the IPA system searched for pathway crosstalk analysis and significant molecular networks. A total of 5 significant molecular networks were identified by Fisher's exact test in the IPA system with additional criteria specifying that a pathway's score was at least 20 and each pathway had at least 10 molecules (Table 10). FIG. 3 showed the most significant network, in which molecules implicated are highlighted in red and green. In this network (Table 10; FIG. 4A), we observed 40 ASD miRNAs candidates, enriched with the functions of neurological and psychological disorders. We highlighted Phosphatase and tensin homolog protein (PTEN) and B-Raf Proto-Oncogene, Serine/Threonine kinase (BRAF) previously described to be regulated by these miRNAs. PTEN and BRAF are essential in synaptic transmission and plasticity, neuronal function, and development of learning/memory. This result is consistent with prior knowledge of ASD phenotypes, providing further evidence of this disorder's neuro-related processes.

In addition to the significant network, there are other crosstalk networks and predicted molecules that are noteworthy (Table 10, FIG. 4B). The most interesting one is Epidermal Growth Factor Receptor (EGFR) associated with symptom severity in children with ASD. Also, Insulin-Like Growth Factor (1IGF-1) is a neurotrophic polypeptide crucial in central nervous system growth, development, and maturation. IGF-1 has emerged as a potential therapeutic approach for several neurodevelopmental disorders and ASD. In children with ASD, stimulation with TLR2 led to a high proinflammatory response. ASD pathogenesis and symptom severity are thought to arise from complex interactions, including immune-inflammatory pathways and mitochondrial dysfunctions.

### Example 5

### Profiling of Plasma PiRNAs in ASD Subjects

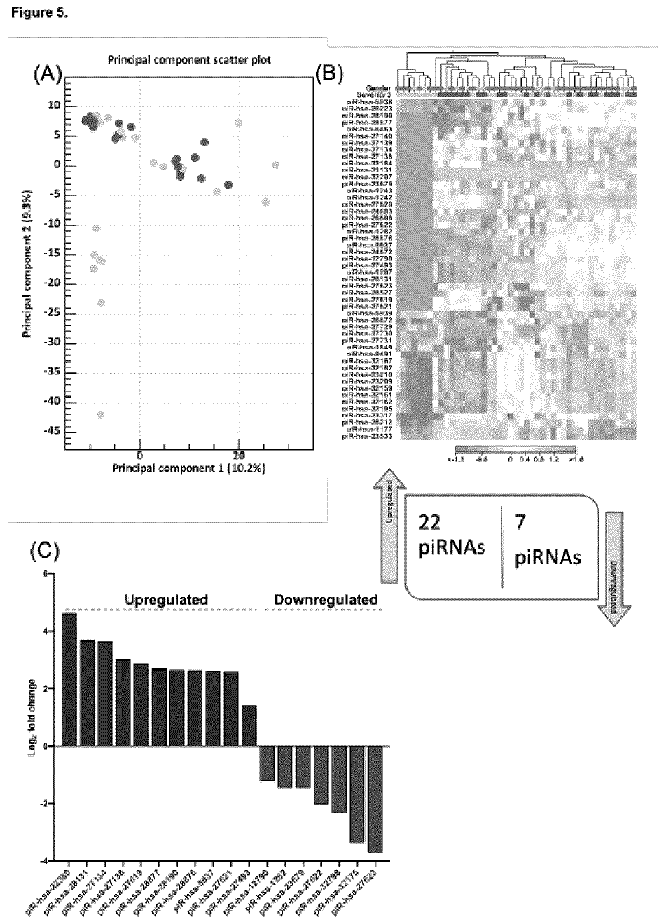

To assign reads to other small RNAs such as piRNAs, the reads were mapped to piRNABank database Human_piRNA_sequence_v1.0 (http://regulatoryrna.org/database/piRNA/download.html). A principal component analysis (PCA) of the piRNAs from each sample demonstrates that samples seemed to cluster primarily by ASD symptomatology; severe and mild symptoms (FIG. 5A). Among the 23,439 piRNAs species in the human genome, the differentially expressed piRNAs between the severe vs. mild groups we selected according to the following criteria: 1) the RPM (the number of reads per million clean tags) values were larger than 50; 2) piRNAs should have at least a 2-fold difference in expression between the groups; 3) p-value<0.05.

As a result, 29 piRNAs were obtained based on these criteria, as shown in the hierarchical clustering analysis of piRNA expression profile (FIG. 5B and Table 10). Furthermore, 22 piRNAs were more expressed within the severe group, and 7 were down-regulated. piR-hsa-28131 is the most up-regulated piRNA (log2FC=3.69) and piR-hsa-27623 is the most down-regulated piRNA (log2FC=−3.70) (FIG. 5B and Table 11).

TABLE 11
Differentially expressed piRNAs (N = 29; Absolute fold
change < 2; p < 0.05). Increased expression in
severe ASD as compared to mild.

| | | Max group | Log₂ fold | Fold | |
|---|---|---|---|---|---|
| | Name | mean | change | change | p-value |
| 1 | piR-hsa-22380 | 405.03 | 4.63 | 24.73 | 0 |
| 2 | piR-hsa-28131 | 225.52 | 3.69 | 12.88 | 0 |
| 3 | piR-hsa-27134 | 238.04 | 3.65 | 12.59 | 0 |
| 4 | piR-hsa-27138 | 102.46 | 3.03 | 8.2 | 0 |
| 5 | piR-hsa-27619 | 84.91 | 2.87 | 7.32 | 8.80E−08 |
| 6 | piR-hsa-28877 | 610.57 | 2.7 | 6.51 | 3.30E−08 |
| 7 | piR-hsa-28190 | 114.61 | 2.66 | 6.34 | 5.40E−07 |
| 8 | piR-hsa-28876 | 107.75 | 2.65 | 6.26 | 0 |
| 9 | piR-hsa-5937 | 146.18 | 2.63 | 6.2 | 0.01 |
| 10 | piR-hsa-27621 | 97.82 | 2.58 | 5.98 | 0.01 |
| 11 | piR-hsa-24683 | 65.41 | 2.53 | 5.77 | 0.01 |
| 12 | piR-hsa-26508 | 60.87 | 2.22 | 4.67 | 0.02 |
| 13 | piR-hsa-27140 | 142.01 | 2.17 | 4.51 | 0.02 |
| 14 | piR-hsa-6463 | 58.64 | 2.14 | 4.41 | 0.01 |
| 15 | piR-hsa-27620 | 67.19 | 2.09 | 4.26 | 0.02 |
| 16 | piR-hsa-32207 | 73.02 | 2.08 | 4.23 | 0.02 |
| 17 | piR-hsa-24672 | 79.74 | 2.05 | 4.14 | 0.03 |
| 18 | piR-hsa-32221 | 250.51 | 2 | 3.99 | 0 |
| 19 | piR-hsa-1242 | 102.28 | 1.92 | 3.78 | 0.02 |
| 20 | piR-hsa-32184 | 682.64 | 1.63 | 3.1 | 0.04 |
| 21 | piR-hsa-1243 | 210.17 | 1.52 | 2.87 | 0.03 |
| 22 | piR-hsa-27493 | 313.6 | 1.42 | 2.68 | 0.04 |
| 23 | piR-hsa-12790 | 224.19 | −1.24 | −2.36 | 0.02 |
| 24 | piR-hsa-1282 | 27311.52 | −1.47 | −2.76 | 0.01 |
| 25 | piR-hsa-23679 | 372.93 | −1.47 | −2.77 | 0.01 |
| 26 | piR-hsa-27622 | 49.07 | −2.04 | −4.1 | 0.02 |
| 27 | piR-hsa-32798 | 150.66 | −2.34 | −5.05 | 0.01 |
| 28 | piR-hsa-32175 | 79.96 | −3.38 | −10.44 | 0 |
| 29 | piR-hsa-27623 | 153.51 | −3.7 | −12.98 | 0 |

### Example 6

### Other RNAs Expression: Y-RNAs and SnoRNAs

The unmapped reads from the QIAseq miRNA quantification workflow were collected and remapped to the full human genome using RNA-seq analysis in CLC Genomics Workbench to assign reads to other noncoding RNAs such as Y-RNAs and snoRNAs. Initially, we compared the expression of Y-RNAs between both groups (22 subjects with severe symptoms vs. 23 subjects with mild symptoms) and identified one Y-RNA; RNY3 (RNA, Ro60-Associated Y3), and three differentially expressed RNY3 and RNY4 pseudogenes; RNY3P1, RNY4P28, and RNY4P29, selected based on absolute fold-change >2 and p-value 0.05 (Table 12). Expression levels of RNY4 pseudogene 29 (RNY4P29) expression levels were significantly higher within the severe group compared to mild, whereas RNY3, RNY3P1, and RNY4P28 were significantly lower in the severe subjects.

Furthermore, according to our analysis, 19 snoRNAs revealed greater expression in severe subjects' plasma, while 4 were downregulated. SNORA69 (also known as U69) was identified to be the most up-regulated snoRNA (logFC=4.63) and SNORD42A (U42) the most down-regulated (logFC=−3.70).

TABLE 12
Differentially expressed Y-RNAs (N = 4) and snoRNAs (N = 23) (Absolute
fold change >2; p < 0.05). Increased expression in severe ASD as compared to mild.

| | | | | | | |
|---|---|---|---|---|---|---|
| | | | Max group | Log₂ fold | Fold | p- |
| Name | Ch * | Region | mean | change | change | value |

Y-RNA

| | | | | | | | |
|---|---|---|---|---|---|---|---|
| 1 | RNY3P1 | 5 | 79170234 . . . 79170335 | 224.19 | −1.24 | −2.36 | 0.02 |
| 2 | RNY3 | 7 | 148983755 . . . 148983856 | 27311.52 | −1.47 | −2.76 | 0.01 |
| 3 | RNY4P29 | 13 | 58527655 . . . 58527751 | 102.46 | 3.03 | 8.20 | 0.00 |
| 4 | RNY4P28 | 13 | 60187738 . . . 60187830 | 372.93 | −1.47 | −2.77 | 0.01 |

snoRNA

| | | | | | | | |
|---|---|---|---|---|---|---|---|
| 1 | SNORA73A | 1 | 28507366 . . . 28507571 | 58.64 | 2.14 | 4.41 | 0.01 |
| 2 | SNORD46 | 1 | 44776490 . . . 44776593 | 42.20 | −1.85 | −3.61 | 0.02 |
| 3 | SNORA41 | 2 | 206162228 . . . 206162359 | 225.52 | 3.69 | 12.88 | 0.00 |
| 4 | SNORA62 | 3 | 39411054 . . . 39411206 | 73.02 | 2.08 | 4.23 | 0.02 |
| 5 | SNORD2 | 3 | 186784796 . . . 186784864 | 313.60 | 1.42 | 2.68 | 0.04 |
| 6 | SNORA63 | 3 | 186787300 . . . 186787431 | 29.43 | 1.72 | 3.30 | 0.05 |
| 7 | SNORA26 | 4 | 52713249 . . . 52713370 | 250.51 | 2.00 | 3.99 | 0.00 |
| 8 | SNORD73A | 4 | 151103827 . . . 151103891 | 79.96 | −3.38 | −10.44 | 0.00 |
| 9 | SNORD72 | 5 | 40832656 . . . 40832735 | 142.01 | 2.17 | 4.51 | 0.02 |
| 10 | SNORA13 | 5 | 112161485 . . . 112161617 | 107.75 | 2.65 | 6.26 | 0.00 |
| 11 | SNORA74D | 5 | 139276180 . . . 139276320 | 60.87 | 2.22 | 4.67 | 0.02 |
| 12 | SNORD101 | 6 | 132815307 . . . 132815379 | 682.64 | 1.63 | 3.10 | 0.04 |
| 13 | SNORD36B | 9 | 133350095 . . . 133350168 | 210.17 | 1.52 | 2.87 | 0.03 |
| 14 | SNORA52 | 11 | 811681 . . . 811814 | 65.41 | 2.53 | 5.77 | 0.01 |
| 15 | SNORA28 | 14 | 103337849 . . . 103337974 | 97.82 | 2.58 | 5.98 | 0.01 |
| 16 | SNORD60 | 16 | 2155023 . . . 2155105 | 146.18 | 2.63 | 6.20 | 0.01 |
| 17 | SNORA46 | 16 | 58548499 . . . 58548633 | 238.04 | 3.65 | 12.59 | 0.00 |
| 18 | SNORD111 | 16 | 70529509 . . . 70529588 | 67.19 | 2.09 | 4.26 | 0.02 |
| 19 | SNORD42A | 17 | 28723429 . . . 28723492 | 153.51 | −3.70 | −12.98 | 0.00 |

* Chromosome

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

Disclosed embodiments comprise:

Embodiment 1. A method of determining a circulating noncoding RNA (cir-ncRNA) profile in a child potentially having autism spectrum disorder, comprising;
quantitating the level of multiple cir-ncRNA from a predetermined panel of cir-ncRNA in a plasma sample from the child, wherein the cir-ncRNA are miRNA, piRNA, Y-RNA, snoRNA, or a combination thereof.

Embodiment 2. A method of diagnosing or stratifying autism spectrum disorder in a potentially affected child, comprising;
quantitating the level of multiple cir-ncRNA from a predetermined panel of cir-ncRNA in a plasma sample from the child, wherein the cir-ncRNA are miRNA, piRNA, Y-RNA, snoRNA, or a combination thereof.

Embodiment 3. The method of embodiment 2, further comprising matching the levels of the panel cir-ncRNA to an ASD-associated cir-ncRNA profile.

Embodiment 4. The method of embodiment 3, wherein the ASD-associated cir-ncRNA profile is associated with severe ASD.

Embodiment 5. The method of embodiment 3, wherein the ASD-associated cir-ncRNA profile is associated with mild ASD.

Embodiment 6. The method of any one of embodiments 1-5 wherein the quantitating is by deep sequencing.

Embodiment 7. The method of embodiment 6, wherein the level of each cir-ncRNA is expressed in reads per million (RPM).

Embodiment 8. The method of claim any one of embodiments 1-7, wherein cir-ncRNA, or cDNA made from the cir-ncRNA, is fractionated by size and a size fraction corresponding to the biotype(s) of the cir-ncRNA in the panel is selected for analysis.

Embodiment 9. The method of any one of embodiments 1-8, wherein the panel comprises miRNA.

Embodiment 10. The method of embodiment 9, wherein the panel of miRNA comprises hsa-miR-302a-5p, hsa-miR-302c-3p, hsa-miR-302a-3p, hsa-miR-302d-3p, hsa-miR-302b-3p, hsa-miR-302c-5p, hsa-miR-135b-5p, hsa-miR-373-3p, hsa-miR-372-3p, hsa-miR-187-3p, hsa-miR-4745-5p, hsa-miR-184, hsa-miR-219a-5p, hsa-miR-6516-5p, hsa-miR-5189-5p, hsa-miR-378g, hsa-let-7f-2-3p, and hsa-miR-6509-5p.

Embodiment 11. The method of embodiment 10, comprising determining whether;
a. hsa-miR-302a-5p, hsa-miR-302c-3p, hsa-miR-302a-3p, hsa-miR-302d-3p, hsa-miR-302b-3p, hsa-miR-302c-5p, hsa-miR-135b-5p, hsa-miR-373-3p, hsa-miR-372-3p, and hsa-miR-187-3p are present at >300 RPM; and
b. hsa-miR-4745-5p, hsa-miR-184, hsa-miR-219a-5p, hsa-miR-6516-5p, hsa-miR-5189-5p, hsa-miR-378g, hsa-let-7f-2-3p, and hsa-miR-6509-5p are present at <10 RPM.

Embodiment 12. The method of embodiment 11, further comprising treating the child for severe ASD if:
a. hsa-miR-302a-5p, hsa-miR-302c-3p, hsa-miR-302a-3p, hsa-miR-302d-3p, hsa-miR-302b-3p, hsa-miR-302c-5p, hsa-miR-135b-5p, hsa-miR-373-3p, hsa-miR-372-3p, and hsa-miR-187-3p are present at >300 RPM; and
b. hsa-miR-4745-5p, hsa-miR-184, hsa-miR-219a-5p, hsa-miR-6516-5p, hsa-miR-5189-5p, hsa-miR-378g, hsa-let-7f-2-3p, and hsa-miR-6509-5p are present at <10 RPM.

Embodiment 13. The method of any one of embodiments 1-8, wherein the panel comprises piRNA.

Embodiment 14. The method of embodiment 13, where in the panel of piRNA comprises piR-hsa-22380, piR-hsa-28131, piR-hsa-27134, piR-hsa-28877, piR-hsa-32221, piR-hsa-32184, and piR-hsa-27493.

Embodiment 15. The method of embodiment 10, comprising determining whether piR-hsa-22380, piR-hsa-28131, piR-hsa-27134, piR-hsa-28877, piR-hsa-32221, piR-hsa-32184, and piR-hsa-27493 are present at >200 RPM.

Embodiment 16. The method of embodiment 15, further comprising treating the child for severe ASD if piR-hsa-22380, piR-hsa-28131, piR-hsa-27134, piR-hsa-28877, piR-hsa-32221, piR-hsa-32184, and piR-hsa-27493 are present at >200 RPM.

Embodiment 17. The method of any one of embodiments 1-8, wherein the panel comprises Y-RNA and/or snoRNA.

Embodiment 18. The method of embodiment 17, where in the panel of Y-RNA and/or snoRNA comprises RNY4P29, SNORD2, SNORD101, SNORA46, and SNORA69.

Embodiment 19. The method of embodiment 18, comprising determining whether:
a. RNY4P29 is present at >100 RPM; and
b. SNORD2, SNORD101, SNORA46, and SNORA69are present at >200 RPM.

Embodiment 20. The method of embodiment 19, further comprising treating the child for severe ASD if:
a. RNY4P29 is present at >100 RPM; and
b. SNORD2, SNORD101, SNORA46, and SNORA69are present at >200 RPM.

Embodiment 21. The method of any one of embodiments 1-20 wherein the child is ≤10 years of age.

Embodiment 22. The method of any one of embodiments 1-20 wherein the child is ≤9 years of age.

Embodiment 23. The method of any one of embodiments 1-20 wherein the child is ≤8 years of age.

Embodiment 24. The method of any one of embodiments 1-20 wherein the child is ≤7 years of age.

Embodiment 25. The method of any one of embodiments 1-20 wherein the child is ≤6 years of age.

Embodiment 26. The method of embodiment 21, wherein the child is from 5-10 years of age.

Embodiment 27. The method of embodiment 22, wherein the child is from 6-9 years of age.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of determining a circulating noncoding RNA (cir-ncRNA) profile in a child potentially having autism spectrum disorder, comprising:
quantitating the level of multiple cir-ncRNA from a predetermined panel of cir-ncRNA in a plasma sample from the child, wherein the cir-ncRNA are miRNA, piRNA, Y-RNA, snoRNA, or a combination thereof.

2. A method of diagnosing or stratifying autism spectrum disorder in a potentially affected child, comprising:
quantitating the level of multiple cir-ncRNA from a predetermined panel of cir-ncRNA in a plasma sample from the child, wherein the cir-ncRNA are miRNA, piRNA, Y-RNA, snoRNA, or a combination thereof.

3. The method of claim 2, further comprising matching the levels of the panel cir-ncRNA to an ASD-associated cir-ncRNA profile.

4. The method of claim 3, wherein the ASD-associated cir-ncRNA profile is associated with severe ASD or mild ASD.

5. (canceled)

6. The method of claim 1 wherein the quantitating is by deep sequencing.

7. The method of claim 6, wherein the level of each cir-ncRNA is expressed in reads per million (RPM).

8. The method of claim 1, wherein cir-ncRNA, or cDNA made from the cir-ncRNA, is fractionated by size and a size fraction corresponding to the biotype(s) of the cir-ncRNA in the panel is selected for analysis.

9. The method of claim 1, wherein the panel comprises miRNA, piRNA, Y-RNA, snoRNA, or a combination thereof.

10. The method of claim 9, wherein the panel of miRNA comprises hsa-miR-302a-5p, hsa-miR-302c-3p, hsa-miR-302a-3p, hsa-miR-302d-3p, hsa-miR-302b-3p, hsa-miR-302c-5p, hsa-miR-135b-5p, hsa-miR-373-3p, hsa-miR-372-3p, hsa-miR-187-3p, hsa-miR-4745-5p, hsa-miR-184, hsa-miR-219a-5p, hsa-miR-6516-5p, hsa-miR-5189-5p, hsa-miR-378g, hsa-let-7f-2-3p, and hsa-miR-6509-5p.

11. The method of claim 10, comprising determining whether:
a) hsa-miR-302a-5p, hsa-miR-302c-3p, hsa-miR-302a-3p, hsa-miR-302d-3p, hsa-miR-302b-3p, hsa-miR-302c-5p, hsa-miR-135b-5p, hsa-miR-373-3p, hsa-miR-372-3p, and hsa-miR-187-3p are present at >300 RPM; and
b) hsa-miR-4745-5p, hsa-miR-184, hsa-miR-219a-5p, hsa-miR-6516-5p, hsa-miR-5189-5p, hsa-miR-378g, hsa-let-7f-2-3p, and hsa-miR-6509-5p are present at <10 RPM.

12. The method of claim 11, further comprising treating the child for severe ASD if:
a) hsa-miR-302a-5p, hsa-miR-302c-3p, hsa-miR-302a-3p, hsa-miR-302d-3p, hsa-miR-302b-3p, hsa-miR-302c-5p, hsa-miR-135b-5p, hsa-miR-373-3p, hsa-miR-372-3p, and hsa-miR-187-3p are present at >300 RPM; and
b) hsa-miR-4745-5p, hsa-miR-184, hsa-miR-219a-5p, hsa-miR-6516-5p, hsa-miR-5189-5p, hsa-miR-378g, hsa-let-7f-2-3p, and hsa-miR-6509-5p are present at <10 RPM.

13. (canceled)

14. The method of claim 13, where in the panel of piRNA comprises piR-hsa-22380, piR-hsa-28131, piR-hsa-27134, piR-hsa-28877, piR-hsa-32221, piR-hsa-32184, and piR-hsa-27493.

15. The method of claim 10, comprising determining whether piR-hsa-22380, piR-hsa-28131, piR-hsa-27134, piR-hsa-28877, piR-hsa-32221, piR-hsa-32184, and piR-hsa-27493 are present at >200 RPM.

16. The method of claim 15, further comprising treating the child for severe ASD if piR-hsa-22380, piR-hsa-28131, piR-hsa-27134, piR-hsa-28877, piR-hsa-32221, piR-hsa-32184, and piR-hsa-27493 are present at >200 RPM.

17. (canceled)

18. The method of claim 17, where in the panel of Y-RNA and/or snoRNA comprises RNY4P29, SNORD2, SNORD101, SNORA46, and SNORA69.

19. The method of claim 18, comprising determining whether:
a) RNY4P29 is present at >100 RPM; and
b) SNORD2, SNORD101, SNORA46, and SNORA69are present at >200 RPM.

20. The method of claim 19, further comprising treating the child for severe ASD if:
a) RNY4P29 is present at >100 RPM; and
b) SNORD2, SNORD101, SNORA46, and SNORA69are present at >200 RPM.

21. The method of claim 1 wherein the child is ≤10 years of age, <9 years of age, ≤8 years of age, ≤7 years of age, or ≤6 years of age.

22-25. (canceled)

26. The method of claim 21, wherein the child is from 5-10 years of age.

27. The method of claim 22, wherein the child is from 6-9 years of age.


SEQUENCE LISTING (115 sequences)

SEQ ID NO: 1 - RNA, 7 nt, Artificial Sequence
  miRNA hsa-miR-302a-5p
cuuaaac

SEQ ID NO: 2 - RNA, 7 nt, Artificial Sequence
  miRNA miR-291a-3p
aagugcu

SEQ ID NO: 3 - RNA, 7 nt, Artificial Sequence
  miRNA miR-302c-5p
uuaacau

SEQ ID NO: 4 - RNA, 7 nt, Artificial Sequence
  miRNA miR-135a-5p
auggcuu

SEQ ID NO: 5 - RNA, 7 nt, Artificial Sequence
  miRNA miR-187-3p
cgugucu

SEQ ID NO: 6 - RNA, 7 nt, Artificial Sequence
  miRNA miR-302b-5p
cuuuaac

SEQ ID NO: 7 - RNA, 7 nt, Artificial Sequence
  miRNA miR-100-3p
aagcuug

SEQ ID NO: 8 - RNA, 7 nt, Artificial Sequence
  miRNA miR-12135
aaagguu

SEQ ID NO: 9 - RNA, 7 nt, Artificial Sequence
  miRNA miR-293-5p
cucaaac

SEQ ID NO: 10 - RNA, 7 nt, Artificial Sequence
  miRNA miR-518a-3p
aaagcgc

SEQ ID NO: 11 - RNA, 7 nt, Artificial Sequence
  miRNA miR-515-5p
ucuccaa

SEQ ID NO: 12 - RNA, 7 nt, Artificial Sequence
  miRNA miR-31-3p
gcuaugc

SEQ ID NO: 13 - RNA, 7 nt, Artificial Sequence
  miRNA miR-302c-3p
agugcuu

SEQ ID NO: 14 - RNA, 7 nt, Artificial Sequence
  miRNA miR-516a-5p
ucucgag

SEQ ID NO: 15 - RNA, 7 nt, Artificial Sequence
  miRNA miR-2682-3p
gccucuu

SEQ ID NO: 16 - RNA, 7 nt, Artificial Sequence
  miRNA miR-1298-5p
ucauucg

SEQ ID NO: 17 - RNA, 7 nt, Artificial Sequence
  miRNA miR-31-5p
ggcaaga

SEQ ID NO: 18 - RNA, 7 nt, Artificial Sequence
  miRNA miR-376a-3p
ucauaga

SEQ ID NO: 19 - RNA, 7 nt, Artificial Sequence
  miRNA miR-106a-3p
ugcaaug

SEQ ID NO: 20 - RNA, 7 nt, Artificial Sequence
  miRNA miR-376b-5p
guggaua

SEQ ID NO: 21 - RNA, 7 nt, Artificial Sequence
  miRNA miR-517a-3p
ucgugca

SEQ ID NO: 22 - RNA, 7 nt, Artificial Sequence
  miRNA miR-18a-5p
aaggugc

SEQ ID NO: 23 - RNA, 7 nt, Artificial Sequence
  miRNA miR-4639-5p
ugcuaag

SEQ ID NO: 24 - RNA, 7 nt, Artificial Sequence
  miRNA miR-105-5p
caaaugc

SEQ ID NO: 25 - RNA, 7 nt, Artificial Sequence
  miRNA miR-3195
gcgccgg

SEQ ID NO: 26 - RNA, 7 nt, Artificial Sequence
  miRNA miR-548o-3p
caaaacu

SEQ ID NO: 27 - RNA, 7 nt, Artificial Sequence
  miRNA miR-138-5p
gcuggug

SEQ ID NO: 28 - RNA, 7 nt, Artificial Sequence
  miRNA miR-143-5p
gugcagu

SEQ ID NO: 29 - RNA, 7 nt, Artificial Sequence
  miRNA miR-34a-5p
ggcagug

SEQ ID NO: 30 - RNA, 7 nt, Artificial Sequence
  miRNA miR-299a-5p
gguuuac

SEQ ID NO: 31 - RNA, 7 nt, Artificial Sequence
  miRNA miR-485-3p
ucauaca

SEQ ID NO: 32 - RNA, 7 nt, Artificial Sequence
  miRNA miR-222-5p
ucaguag

SEQ ID NO: 33 - RNA, 7 nt, Artificial Sequence
  miRNA miR-3168
aguucua

SEQ ID NO: 34 - RNA, 7 nt, Artificial Sequence
  miRNA miR-503-3p
ggguauu

SEQ ID NO: 35 - RNA, 7 nt, Artificial Sequence
  miRNA miR-154-5p
agguuau

SEQ ID NO: 36 - RNA, 7 nt, Artificial Sequence
  miRNA miR-136-5p
cuccauu

SEQ ID NO: 37 - RNA, 7 nt, Artificial Sequence
  miRNA miR-218-5p
ugugcuu

SEQ ID NO: 38 - RNA, 7 nt, Artificial Sequence
  miRNA miR-15a-3p
aggccau

SEQ ID NO: 39 - RNA, 7 nt, Artificial Sequence
  miRNA miR-1587
ugggcug

SEQ ID NO: 40 - RNA, 7 nt, Artificial Sequence
  miRNA miR-199a-5p
ccagugu

SEQ ID NO: 41 - RNA, 7 nt, Artificial Sequence
  miRNA miR-363-5p
gggugga

SEQ ID NO: 42 - RNA, 7 nt, Artificial Sequence
  miRNA miR-122b-3p
aacacca

SEQ ID NO: 43 - RNA, 7 nt, Artificial Sequence
  miRNA miR-17-5p
aaagugc

SEQ ID NO: 44 - RNA, 7 nt, Artificial Sequence
  miRNA miR-16-5p
agcagca

SEQ ID NO: 45 - RNA, 7 nt, Artificial Sequence
  miRNA miR-125b-5p
cccugag

SEQ ID NO: 46 - RNA, 7 nt, Artificial Sequence
  miRNA miR-494-3p
gaaacau

SEQ ID NO: 47 - RNA, 7 nt, Artificial Sequence
  miRNA miR-411-3p
auguaac

SEQ ID NO: 48 - RNA, 7 nt, Artificial Sequence
  miRNA miR-125b-1-3p
cggguua

SEQ ID NO: 49 - RNA, 7 nt, Artificial Sequence
  miRNA miR-455-5p
augugcc

SEQ ID NO: 50 - RNA, 7 nt, Artificial Sequence
  miRNA miR-411-5p
aguagac

SEQ ID NO: 51 - RNA, 7 nt, Artificial Sequence
  miRNA miR-100-5p
acccgua

SEQ ID NO: 52 - RNA, 7 nt, Artificial Sequence
  miRNA miR-542-3p
gugacag

SEQ ID NO: 53 - RNA, 7 nt, Artificial Sequence
  miRNA miR-377-3p
ucacaca

SEQ ID NO: 54 - RNA, 7 nt, Artificial Sequence
  miRNA miR-493-3p
gaagguc

SEQ ID NO: 55 - RNA, 7 nt, Artificial Sequence
  miRNA miR-1277-5p
aauauau

SEQ ID NO: 56 - RNA, 7 nt, Artificial Sequence
  miRNA miR-136-3p
aucaucg

SEQ ID NO: 57 - RNA, 7 nt, Artificial Sequence
  miRNA miR-149-5p
cuggcuc

SEQ ID NO: 58 - RNA, 7 nt, Artificial Sequence
  miRNA miR-145-3p
gauuccu

SEQ ID NO: 59 - RNA, 7 nt, Artificial Sequence
  miRNA miR-145-5p
uccaguu

SEQ ID NO: 60 - RNA, 7 nt, Artificial Sequence
  miRNA miR-3104-5p
agggggc

SEQ ID NO: 61 - RNA, 7 nt, Artificial Sequence
  miRNA miR-1-3p
ggaaugu

SEQ ID NO: 62 - RNA, 7 nt, Artificial Sequence
  miRNA miR-197-5p
ggguaga

SEQ ID NO: 63 - RNA, 7 nt, Artificial Sequence
  miRNA miR-1247-3p
gggaacg

SEQ ID NO: 64 - RNA, 7 nt, Artificial Sequence
  miRNA miR-548l
aaaguau

SEQ ID NO: 65 - RNA, 7 nt, Artificial Sequence
  miRNA miR-181a-5p
acauuca

SEQ ID NO: 66 - RNA, 7 nt, Artificial Sequence
  miRNA miR-412-5p
ggucgac

SEQ ID NO: 67 - RNA, 7 nt, Artificial Sequence
  miRNA miR-548h-5p
aaaguaa

SEQ ID NO: 68 - RNA, 7 nt, Artificial Sequence
  miRNA miR-3150b-3p
gaggaga

SEQ ID NO: 69 - RNA, 7 nt, Artificial Sequence
  miRNA miR-12118
aaggagg

SEQ ID NO: 70 - RNA, 7 nt, Artificial Sequence
  miRNA miR-151-5p
cgaggag

SEQ ID NO: 71 - RNA, 7 nt, Artificial Sequence
  miRNA miR-216a-5p
aaucuca

SEQ ID NO: 72 - RNA, 7 nt, Artificial Sequence
  miRNA miR-202-5p
uccuaug

SEQ ID NO: 73 - RNA, 7 nt, Artificial Sequence
  miRNA miR-378a-3p
cuggacu

SEQ ID NO: 74 - RNA, 7 nt, Artificial Sequence
  miRNA miR-149-3p
gggaggg

SEQ ID NO: 75 - RNA, 7 nt, Artificial Sequence
  miRNA miR-149-3p
gugggac

SEQ ID NO: 76 - RNA, 7 nt, Artificial Sequence
  miRNA miR-450b-3p
uugggga

SEQ ID NO: 77 - RNA, 7 nt, Artificial Sequence
  miRNA miR-4481
gaguggg

SEQ ID NO: 78 - RNA, 7 nt, Artificial Sequence
  miRNA miR-184
ggacgga

SEQ ID NO: 79 - RNA, 7 nt, Artificial Sequence
  miRNA miR-219a-5p
gauuguc

SEQ ID NO: 80 - RNA, 7 nt, Artificial Sequence
  miRNA miR-128-1-5p
ggggccg

SEQ ID NO: 81 - RNA, 7 nt, Artificial Sequence
  miRNA miR-6516-5p
uugcagu

SEQ ID NO: 82 - RNA, 7 nt, Artificial Sequence
  miRNA miR-1285-3p
cugggca

SEQ ID NO: 83 - RNA, 7 nt, Artificial Sequence
  miRNA miR-378g
cugggcu

SEQ ID NO: 84 - RNA, 7 nt, Artificial Sequence
  miRNA let-7f-2-3p
uauacag

SEQ ID NO: 85 - RNA, 7 nt, Artificial Sequence
  miRNA miR-6509-5p
uuaggua

SEQ ID NO: 86 - RNA, 23 nt, Artificial Sequence
  miRNA hsa-miR-302a-5p
acuuaaacguggauguacuugcu

SEQ ID NO: 87 - RNA, 23 nt, Artificial Sequence
  miRNA hsa-miR-302c-3p
uaagugcuuccauguuucagugg

SEQ ID NO: 88 - RNA, 23 nt, Artificial Sequence
  miRNA hsa-miR-302a-3p
uaagugcuuccauguuuugguga

SEQ ID NO: 89 - RNA, 23 nt, Artificial Sequence
  miRNA hsa-miR-302d-3p
uaagugcuuccauguuugagugu

SEQ ID NO: 90 - RNA, 23 nt, Artificial Sequence
  miRNA hsa-miR-302b-3p
uaagugcuuccauguuuuaguag

SEQ ID NO: 91 - RNA, 22 nt, Artificial Sequence
  miRNA hsa-miR-302c-5p
uuuaacauggggguaccugcug

SEQ ID NO: 92 - RNA, 23 nt, Artificial Sequence
  miRNA hsa-miR-135b-5p
uauggcuuuucauuccuauguga

SEQ ID NO: 93 - RNA, 23 nt, Artificial Sequence
  miRNA hsa-miR-373-3p
gaagugcuucgauuuuggggugu

SEQ ID NO: 94 - RNA, 23 nt, Artificial Sequence
  miRNA hsa-miR-372-3p
aaagugcugcgacauuugagcgu

SEQ ID NO: 95 - RNA, 22 nt, Artificial Sequence
  miRNA hsa-miR-187-3p
ucgugucuuguguugcagccgg

SEQ ID NO: 96 - RNA, 23 nt, Artificial Sequence
  miRNA hsa-miR-4745-5p
ugaguggggcucccgggacggcg

SEQ ID NO: 97 - RNA, 22 nt, Artificial Sequence
  miRNA hsa-miR-184
uggacggagaacugauaagggu

SEQ ID NO: 98 - RNA, 21 nt, Artificial Sequence
  miRNA hsa-miR-219a-5p
ugauuguccaaacgcaauucu

SEQ ID NO: 99 - RNA, 22 nt, Artificial Sequence
  miRNA hsa-miR-6516-5p
uuugcaguaacaggugugagca

SEQ ID NO: 100 - RNA, 24 nt, Artificial Sequence
  miRNA hsa-miR-5189-5p
ucugggcacaggcggauggacagg

SEQ ID NO: 101 - RNA, 20 nt, Artificial Sequence
  miRNA hsa-miR-378g
acugggcuuggagucagaag

SEQ ID NO: 102 - RNA, 22 nt, Artificial Sequence
  miRNA hsa-let-7f-2-3p
cuauacagucuacugucuuucc

SEQ ID NO: 103 - RNA, 21 nt, Artificial Sequence
  miRNA hsa-miR-6509-5p
auuagguaguggcaguggaac

SEQ ID NO: 104 - DNA, 27 bp, Artificial Sequence
  piRNA piR-hsa-22380
tgttaaccgaaagattggtggttcgag

SEQ ID NO: 105 - DNA, 31 bp, Artificial Sequence
  piRNA piR-hsa-28131
ggcattggtggttcagtggtagaattctcgc

SEQ ID NO: 106 - DNA, 31 bp, Artificial Sequence
  piRNA piR-hsa-27134
gcctggatagctcagttggtagagcatcaga

SEQ ID NO: 107 - DNA, 32 bp, Artificial Sequence
  piRNA piR-hsa-28877
gtttccgtagtgtagtggtcatcacgttcgcc

SEQ ID NO: 108 - DNA, 25 bp, Artificial Sequence
  piRNA piR-hsa-32221
tagtgcgctatgccgatcgggtgtc

SEQ ID NO: 109 - DNA, 25 bp, Artificial Sequence
  piRNA piR-hsa-32184
agtgcgctatgccgatcgggtgtcc

SEQ ID NO: 110 - DNA, 30 bp, Artificial Sequence
  piRNA piR-hsa-27493
gcattggtggttcagtggtagaattctcac

SEQ ID NO: 111 - RNA, 97 nt, Artificial Sequence
  Y-RNA RNY4P29
gguugguccaaugauaguggguuaucagaacuuauuaacauuuagugucacuaaaguugguauaaaacccuccacugcuaaauuuaaaugguuuaua

SEQ ID NO: 112 - RNA, 69 nt, Artificial Sequence
  snoRNA SNORD2
aagugaaaugauggcaaucaucuuucgggacugaccugaaaugaagagaauacucauugcugaucacuu

SEQ ID NO: 113 - RNA, 73 nt, Artificial Sequence
  snoRNA SNORD101
guuugaaugaugacuuuaauugucggauaccccuucacuccuuuuaugagugaaacauaagagucugacaaac

SEQ ID NO: 114 - RNA, 135 nt, Artificial Sequence
  snoRNA SNORA46
agcacuauauuuaaaccuguggaugggaauauuccccauucuugguuacgcuguagugcaaaagaauuccuggcucucuguugcacagcugacuugugccauucugcuguugcuguauagaguuaaggaacaugg

SEQ ID NO: 115 - RNA, 132 nt, Artificial Sequence
  snoRNA SNORA69
aaagcagguugcaauuacagugcuucauuuuguggaaguacugccauuauccugcugaaagaaaagccguguuaaucauuuuugauuuugccuuuaugaggguaaaaucaugacagauugacauggacaauu